(19) United States
(12) Patent Application Publication
(10) Pub. No.: US 2023/0040185 A1
(43) Pub. Date: Feb. 9, 2023

(54) A TIME-SENSITIVE TRIGGER FOR A STREAMING DATA ENVIRONMENT

(71) Applicant: PRENOSIS, INC., Chicago, IL (US)

(72) Inventors: Ishan Taneja, Chicago, IL (US); Carlos G. Lopez-Espina, Evanston, IL (US); Sihai Dave Zhao, Champaign, IL (US); Ruoqing Zhu, Savoy, IL (US); Bobby Reddy, Jr., Chicago, IL (US)

(21) Appl. No.: 17/791,879

(22) PCT Filed: Jan. 12, 2021
(86) PCT No.: PCT/US21/13141

§ 371 Date: Jul. 8, 2022

Related U.S. Application Data
(60) Provisional application No. 62/959,742, filed on Jan. 10, 2020.

(51) Int. Cl. G16H 50/30

(52) U.S. Cl. CPC G16H 50/30

(57) ABSTRACT

A method for making dynamic risk predictions is provided. The method includes receiving a dataset with a first data field and a second data field. The first data field is populated with a measured value. The method also includes imputing a first predicted value to the second data field, generating a first risk score and a first set of associated metrics based on the measured value and the first predicted value, and imputing a second predicted value to the second data field. The method also includes calculating a statistically derived metric and determining whether the statistically derived metric exceeds a predetermined threshold, wherein a predetermined action is recommended if the statistically derived metric exceeds the predetermined threshold. A system and a non-transitory, computer readable medium storing instructions to cause the system to perform the above method are also provided.

### CROSS REFERENCE TO RELATED APPLICATIONS

The present application claims priority to and the benefit of the U.S. Provisional Patent Application No. 62/959,742, filed Jan. 10, 2020, titled “Time-Sensitive Trigger for a Streaming Data Environment,” which is hereby incorporated by reference in its entirety as if fully set forth below and for all applicable purposes.

### FIELD OF THE DISCLOSURE

The present disclosure generally relates to a time-sensitive trigger engine operating in a streaming data environment. More specifically, the present disclosure relates to devices in the healthcare industry that help healthcare personnel make time-sensitive decisions rapidly from incomplete data instances with a high confidence level.

### INTRODUCTION

Predictive models often face the challenge of missing data when deployed in real-world environments. Traditional solutions to this problem generally employ some method to impute missing data so the model can generate an output. However, an added dimension of complexity is introduced in a time-sensitive, streaming data environment where different parameters, each with varying importance, arrive at different times. In such a situation, merely waiting for all the parameters used by the model to arrive is generally suboptimal from the standpoint of outputting accurate predictions as early as possible. Such applications may occur in emergency situations: for urgent care or medical attention, or in other environments such as stock investment decisions and other financial configurations. By the same token, in the above situations it is desirable to obtain an early and accurate prediction of an outcome based on input data that may be incomplete.

### SUMMARY

In some embodiments, a method for making dynamic risk predictions includes receiving a dataset including a first data field and a second data field, wherein the first data field is populated with a measured value. The method also includes imputing a first predicted value to the second data field, generating a first risk score and a first set of associated metrics based on the measured value and the first predicted value, and imputing a second predicted value to the second data field. The method also includes generating a second risk score and a second set of associated metrics based on the measured value and the second predicted value, and calculating a statistically derived metric based on the first risk score, the first set of associated metrics, the second risk score, and the second set of associated metrics. The method also includes determining whether the statistically derived metric exceeds a predetermined threshold, wherein a predetermined action is recommended if the statistically derived metric exceeds the predetermined threshold.

In some embodiments, a system includes a memory configured to store instructions and one or more processors communicatively coupled to the memory. The one or more processors are configured to execute the instructions and cause the system to receive a dataset including a first data field and a second data field, wherein the first data field is populated with a measured value. The one or more processors are also configured to impute a first predicted value to the second data field, to generate a first risk score and a first set of associated metrics based on the measured value and the first predicted value, to impute a second predicted value to the second data field, and to generate a second risk score and a second set of associated metrics based on the measured value and the second predicted value. The one or more processors are also configured to calculate a statistically derived metric based on the first risk score, the first set of associated metrics, the second risk score, and the second set of associated metrics, and to determine whether the statistically derived metric exceeds a predetermined threshold, wherein a predetermined action is recommended if the statistically derived metric exceeds the predetermined threshold, wherein generating the first set of associated metrics includes determining a variability induced in the first risk score by the first predicted value in a between standard deviation value.

In some embodiments, a non-transitory, computer readable medium stores instructions which, when executed by a computer, cause the computer to perform a method. The method includes receiving a dataset including a first data field and a second data field, wherein the first data field is populated with a measured value, imputing a first predicted value to the second data field, and generating a first risk score and a first set of associated metrics based on the measured value and the first predicted value. The method also includes imputing a second predicted value to the second data field, generating a second risk score and a second set of associated metrics based on the measured value and the second predicted value, calculating a statistically derived metric based on the first risk score, the first set of associated metrics, the second risk score, and the second set of associated metrics, and determining whether the statistically derived metric exceeds a predetermined threshold, wherein a predetermined action is recommended if the statistically derived metric exceeds the predetermined threshold. Generating the first set of associated metrics includes determining a variability induced in the first risk score by the first predicted value in a between standard deviation value and in a within standard deviation value.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 illustrates an example architecture suitable for a time-sensitive trigger in a streaming data environment, in accordance with various embodiments.

FIG. 2 is a block diagram illustrating an example server and client from the architecture of FIG. 1, according to certain aspects of the disclosure.

FIG. 3 illustrates a block diagram of a trigger system for a time-sensitive, streaming data environment, in accordance with various embodiments.

FIG. 4 illustrates a block diagram of a trigger logic input generator for a trigger system, in accordance with various embodiments.

FIG. 5 illustrates an exemplary table of a dataset including a time sequence of multiple clinical tests for a patient, in accordance with various embodiments.

FIG. 6 illustrates a table indicative of multiple features associated with a patient in a time sequence, and a trigger result for a healthcare action based on the features, in accordance with various embodiments.

FIG. 7 is a partial illustration of an input table associated with features that may trigger an action for a patient in a time sequence, in accordance with various embodiments.

FIG. 8 is a partial illustration of a training dataset, in accordance with various embodiments.

FIG. 9 is a partial illustration of a training dataset with model outputs and standard deviations, in accordance with various embodiments.

FIGS. 10A-10F are graphical illustrations of exemplary trigger logic rules, in accordance with various embodiments.

FIG. 11 illustrates a time sequence of actions triggered by a trigger logic engine with a stateless trigger logic, in accordance with various embodiments.

FIG. 12 illustrates a time sequence of actions triggered by a trigger logic engine with a stateful trigger logic, in accordance with various embodiments.

FIGS. 13A-13B are charts illustrating a time evolution of a standard deviation distribution over a risk factor, in accordance with various embodiments.

FIGS. 14A-14I are charts illustrating a diagnostic performance with a stateless trigger logic engine, in accordance with various embodiments.

FIGS. 15A-15I are charts illustrating a diagnostic performance with a stateful trigger logic engine, in accordance with various embodiments.

FIG. 16 is a chart illustrating a probability to take action for a patient over time based on multiple medical features, in accordance with various embodiments.

FIG. 17 is a bar plot of a risk factor for two different sets of patients over several medical features, in accordance with various embodiments.

FIG. 18 is a flow chart illustrating steps in a method to perform a medical action on a patient based on multiple medical features received or imputed over a time sequence, in accordance with various embodiments.

FIG. 19 is a flow chart illustrating steps in a method to perform a medical action on a patient based on multiple medical features received or imputed over a time sequence, in accordance with various embodiments.

FIG. 20 is a flow chart illustrating steps in a method to perform a medical action on a patient based on multiple medical features received or imputed over a time sequence, in accordance with various embodiments.

FIG. 21 is a block diagram illustrating an example computer system with which the client and server of FIGS. 1 and 2, and the methods of FIGS. 18-20 can be implemented, in accordance with various embodiments.

In the figures, elements and steps denoted by the same or similar reference numerals are associated with the same or similar elements and steps, unless indicated otherwise.

### DETAILED DESCRIPTION

In the following detailed description, numerous specific details are set forth to provide a full understanding of the present disclosure. It will be apparent, however, to one ordinarily skilled in the art, that the embodiments of the present disclosure may be practiced without some of these specific details. In other instances, well-known structures and techniques have not been shown in detail so as not to obscure the disclosure.

### General Overview

Machine learning (ML) models often face the challenge of missing data when deployed in real-world environments. Traditional ML, artificial intelligence (AI), and neural network (NN) algorithms are trained using a large amount of data inputs prior to analysis. Accordingly, systems using any of the above algorithms desirably have complete sets of input data available before evaluation using the trained ML/AI/NN algorithms. However, in a streaming data environment or other time-sensitive configurations, data flows into the system on a streaming basis, typically beyond the control of the system itself. Further, streaming data environments collect information asynchronously, such that different parameters and values, each with varying importance, may be collected into the modeling tool at different times. Accordingly, the problem of performing time-sensitive predictive analysis in a streaming data environment involves optimizing traditional metrics to predict an outcome, e.g., accuracy, sensitivity, specificity, area under the curve for receiver operating characteristics (AUCROC), and the like, in addition to minimizing the time to take a corrective or pre-emptive action (e.g., displaying an output to an end user, manipulating a robot, purchasing a financial instrument, and the like). This is a technical problem originating in the computer field of data analysis to determine predictable outcomes and to take pre-emptive actions accordingly. In various embodiments, a solution to this problem includes methods and systems to impute missing data for a given streaming data instance into a model, computing metrics quantifying the certainty of a corresponding prediction, and feeding such metrics into a rule-based logic system that controls whether or not the system takes an action. In various embodiments, the rule-based logic system can operate in a stateful manner, meaning the system can trigger based on metrics and predictions derived from both current and prior data instances. Embodiments as disclosed herein include frameworks, methods, method evaluation metrics, and secondary applications of such methods to address the challenge of deploying machine learning systems in time-sensitive, streaming data environments.

Embodiments as disclosed herein provide a solution to the above problem in the form of a trigger logic engine that can predict an outcome based on complete or incomplete input data. In various embodiments, the trigger logic engine quantifies the certainty of the predicted outcome, based on the amount of data available (complete/incomplete, or imputed data) and on other statistical values associated with the predicted outcome(s) (e.g., variance, standard deviation and the like). When a metric that is derived from such statistical values is higher than a pre-selected threshold, then the trigger logic engine provides the predicted output (e.g., to a healthcare personnel, or user that may take an action based on the predicted output). In some embodiments, the trigger logic engine may further provide one or more actions recommended (or mandatory), based on the predicted output. When the certainty of the predicted outcome is lower than (or equal to) the pre-selected threshold, the trigger logic engine postpones any action or output until a further time (e.g., when more data is available) and repeats the process.

In accordance to various embodiments, methods and systems consistent with the present disclosure may be applied in the healthcare industry, where medical personnel (e.g., physicians, nurses, paramedics, and the like) may benefit from a low-risk evaluation of an emergency situation, when a medical action may be critical. In various embodiments, methods and systems as disclosed herein may be applied in the financial industry, where large amounts of streaming data (e.g., current and previous stock values of multiple public enterprises) may lead to critical decisions based on the accurate prediction of an outcome.

The proposed solution further provides improvements to the functioning of the computer itself because it saves data storage space and reduces network usage due to the shortened time-to-decision resulting from methods and systems as disclosed herein.

Although many examples provided herein describe a patient's data being identifiable, or download history for images being stored, each user may grant explicit permission for such patient information to be shared or stored. The explicit permission may be granted using privacy controls integrated into the disclosed system. Each user may be provided notice that such patient information can or will be shared with explicit consent, and each patient may at any time end having the information shared, and may delete any stored user information. The stored patient information may be encrypted to protect patient security.

### Example System Architecture

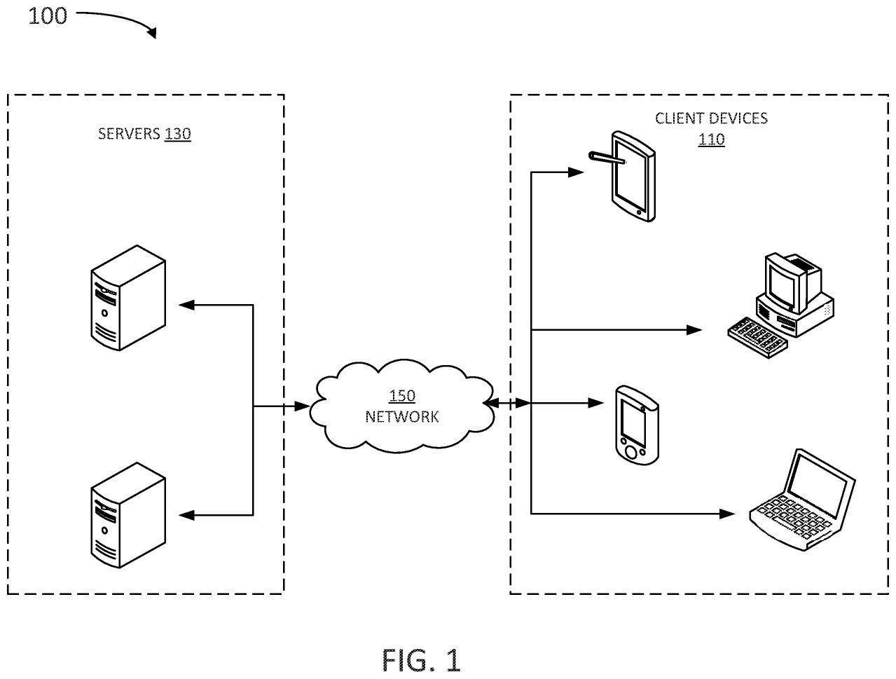

FIG. 1 illustrates an example architecture 100 for a time-sensitive trigger in a streaming data environment, in accordance with various embodiments. Architecture 100 includes servers 130 and client devices 110 connected over a network 150. One of the many servers 130 is configured to host a memory including instructions which, when executed by a processor, cause the server 130 to perform at least some of the steps in methods as disclosed herein. At least one of servers 130 may include, or have access to, a database including clinical data for multiple patients.

Servers 130 may include any device having an appropriate processor, memory, and communications capability for hosting the collection of images and a trigger logic engine. The trigger logic engine may be accessible by various client devices 110 over network 150. Client devices 110 can be, for example, desktop computers, mobile computers, tablet computers (e.g., including e-book readers), mobile devices (e.g., a smartphone or PDA), or any other devices having appropriate processor, memory, and communications capabilities for accessing the trigger logic engine on one of servers 130. In accordance to various embodiments, client devices 110 may be used by healthcare personnel such as physicians, nurses or paramedics, accessing the trigger logic engine on one of servers 130 in a real-time emergency situation (e.g., in a hospital, clinic, ambulance, or any other public or residential environment). In some embodiments, one or more users of client devices 110 (e.g., nurses, paramedics, physicians, and other healthcare personnel) may provide clinical data to the trigger logic engine in one or more server 130, via network 150. In yet other embodiments, one or more client devices 110 may provide the clinical data to server 130 automatically. For example, in some embodiments, client device 110 may be a blood testing unit in a clinic, configured to provide patient results to server 130 automatically, through a network connection. Network 150 can include, for example, any one or more of a local area network (LAN), a wide area network (WAN), the Internet, and the like. Further, network 150 can include, but is not limited to, any one or more of the following network topologies, including a bus network, a star network, a ring network, a mesh network, a star-bus network, tree or hierarchical network, and the like.

### Example Trigger System

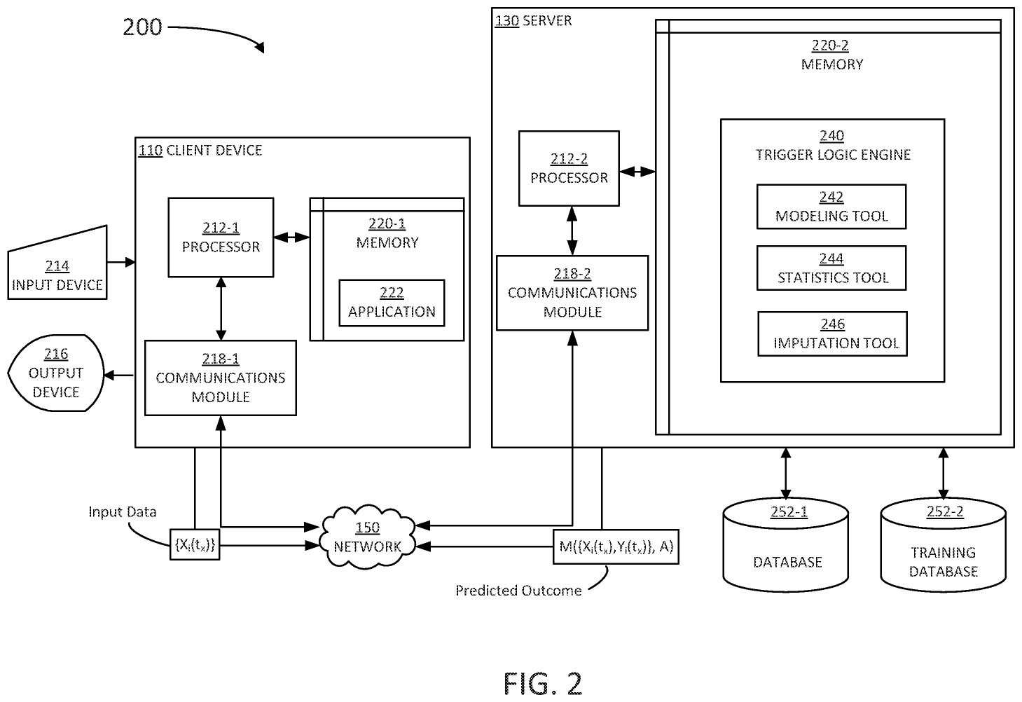

FIG. 2 is a block diagram 200 illustrating an example server 130 and client device 110 in the architecture 100 of FIG. 1, according to certain aspects of the disclosure. Client device 110 and server 130 are communicatively coupled over network 150 via respective communications modules 218-1 and 218-2 (hereinafter, collectively referred to as “communications modules 218”). Communications modules 218 are configured to interface with network 150 to send and receive information, such as data, requests, responses, and commands to other devices on the network. Communications modules 218 can be, for example, modems or Ethernet cards. Client device 110 and server 130 may include a memory 220-1 and 220-2 (hereinafter, collectively referred to as “memories 220”), and a processor 212-1 and 212-2 (hereinafter, collectively referred to as “processors 212”), respectively. Memories 220 may store instructions which, when executed by processors 212, cause either one of client device 110 or server 130 to perform one or more steps in methods as disclosed herein. Accordingly, processors 212 may be configured to execute instructions, such as instructions physically coded into processors 212, instructions received from software in memories 220, or a combination of both.

In accordance with various embodiments, server 130 may include, or be communicatively coupled to, a database 252-1 and a training database 252-2 (hereinafter, collectively referred to as “databases 252”). In one or more implementations, databases 252 may store clinical data for multiple patients. In accordance to various embodiments, training database 252-2 may be the same as database 252-1, or may be included therein. The clinical data in databases 252 may include metrology information such as non-identifying patient characteristics; vital signs; blood measurements such as complete blood count (CBC), comprehensive metabolic panel (CMP), and blood gas (e.g., Oxygen, CO₂, and the like); immunologic information; biomarkers; culture; and the like. The non-identifying patient characteristics may include age, gender, and general medical history, such as a chronic condition (e.g., diabetes, allergies, and the like). In various embodiments, the clinical data may also include actions taken by healthcare personnel in response to metrology information, such as therapeutic measures, medication administration events, dosages, and the like. In various embodiments, the clinical data may also include events and outcomes occurring in the patient's history (e.g., sepsis, stroke, cardiac arrest, shock, and the like). Although databases 252 are illustrated as separated from server 130, in certain aspects, databases 252 and trigger logic engine 240 can be hosted in the same server 130, and be accessible by any other server or client device in network 150.

Memory 220-2 in server 130 may include a trigger logic engine 240 for evaluating a streaming data input and triggering an action based on a predicted outcome thereof. Trigger logic engine 240 may include a modeling tool 242, a statistics tool 244, and an imputation tool 246. Modeling tool 242 may include instructions and commands to collect relevant clinical data and evaluate a probable outcome. Modeling tool 242 may include commands and instructions from a neural network (NN), such as a deep neural network (DNN), a convolutional neural network (CNN), and the like. According to various embodiments, modeling tool 242 may include a machine learning algorithm, an artificial intelligence algorithm, or any combination thereof. Statistics tool 244 evaluates prior data collected by trigger logic engine 240, stored in databases 252, or provided by modeling tool 242. Imputation tool 246 may provide modeling tool 242 with data inputs otherwise missing from a metrology information collected by trigger logic engine 240.

Client device 110 may access trigger logic engine 240 through an application 222 or a web browser installed in client device 110. Processor 212-1 may control the execution of application 222 in client device 110. In accordance to various embodiments, application 222 may include a user interface displayed for the user in an output device 216 of client device 110 (e.g., a graphical user interface—GUI—). A user of client device 110 may use an input device 214 to enter input data as metrology information or to submit a query to trigger logic engine 240 via the user interface of application 222. In accordance with some embodiments, an input data, {Xᵢ(tₓ)}, may be a 1×n vector where Xᵢⱼ indicates, for a given patient, i, a data entry j (0≤j≤n), indicative of any one of multiple clinical data values (or stock prices) that may or may not be available, and tₓ indicates a collection time when the data entry was collected. In some instances, the available clinical data values or stock prices may be measured values (e.g., in contrast to predicted values) populating at least some of the data fields of the input data, {Xᵢ(tₓ)}. Client device 110 may receive, in response to input data {Xᵢ(tₓ)}, a predicted outcome, M({Xᵢ(tₓ), Yᵢ(tₓ)}), from server 130. In accordance to some embodiments, predicted outcome M({Xᵢ(tₓ), Yᵢ(tₓ)}), may be determined based not only on input data, {Xᵢ(tₓ)}, but also on an imputed data, {Yᵢ(tₓ)}. Accordingly, imputed data {Yᵢ(tₓ)} may be provided by imputation tool 246 in response to missing data from the set {Xᵢ(tₓ)}. Input device 214 may include a stylus, a mouse, a keyboard, a touch screen, a microphone, or any combination thereof. Output device 216 may also include a display, a headset, a speaker, an alarm or a siren, or any combination thereof.

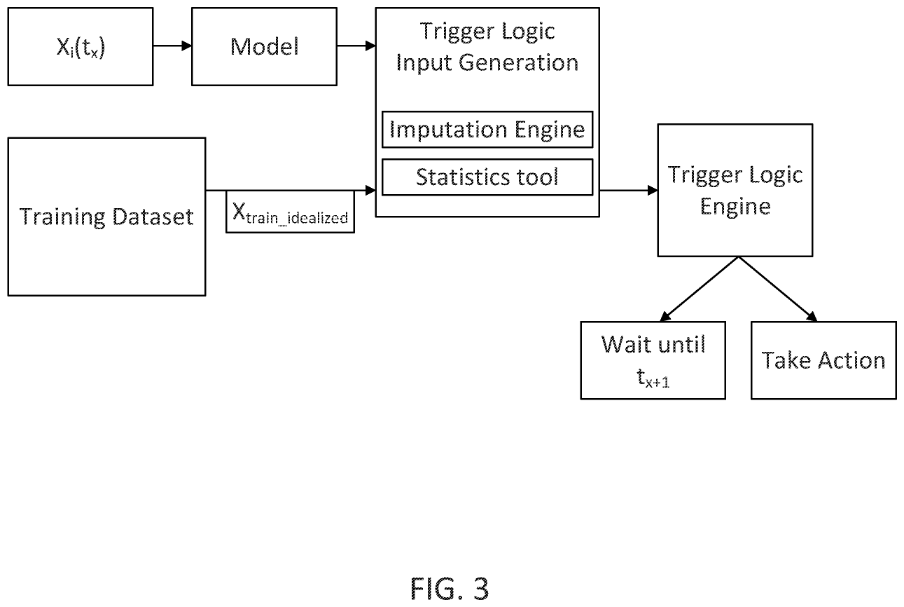

FIG. 3 illustrates a block diagram of a trigger system for a time-sensitive, streaming data environment, in accordance with various embodiments. The trigger system includes a model (hereinafter, designated as M) that provides input data {Xᵢ(tₓ)} to a trigger logic input generation module. The trigger logic input generation module includes an imputation engine and a statistics tool. The imputation engine provides imputed data {Yᵢ(tₓ)}. In accordance to various embodiments, the model may include a machine learning model, and artificial intelligence model, a neural network model or any combination thereof, configured to predict an outcome O using a training dataset (hereinafter, referred to as X_{train_idealized}). In accordance to various embodiments, X_{train_idealized} is an m by n matrix, where m refers to the number of patients and n refers to the number of features in the clinical data that may be relevant to an outcome for each of the patients. In accordance to various embodiments, for each row in X_{train_idealized}, some or all features (e.g., clinical data values) may be available (e.g., measured or otherwise provided by medical personnel, a patient, and the like), regardless of the actual time it is available.

In accordance to various embodiments, M is applied to input {Xᵢ(tₓ)}, wherein the features are assumed to arrive on a streaming basis so, for a given patient i, each feature j arrives at an arbitrary collection time tₓ. For each feature, collection time, tₓ, may be on a pre-determined schedule, asynchronous, or random. The trigger logic engine provides a decision as to whether or not the system should take an action based on metrics (defined later) derived from the statistics tool. In accordance to various embodiments, the trigger logic engine may decide to not take an action at time tₓ, and then the same process is repeated at time tₓ₊₁, when new data Xᵢ(tₓ₊₁) may arrive.

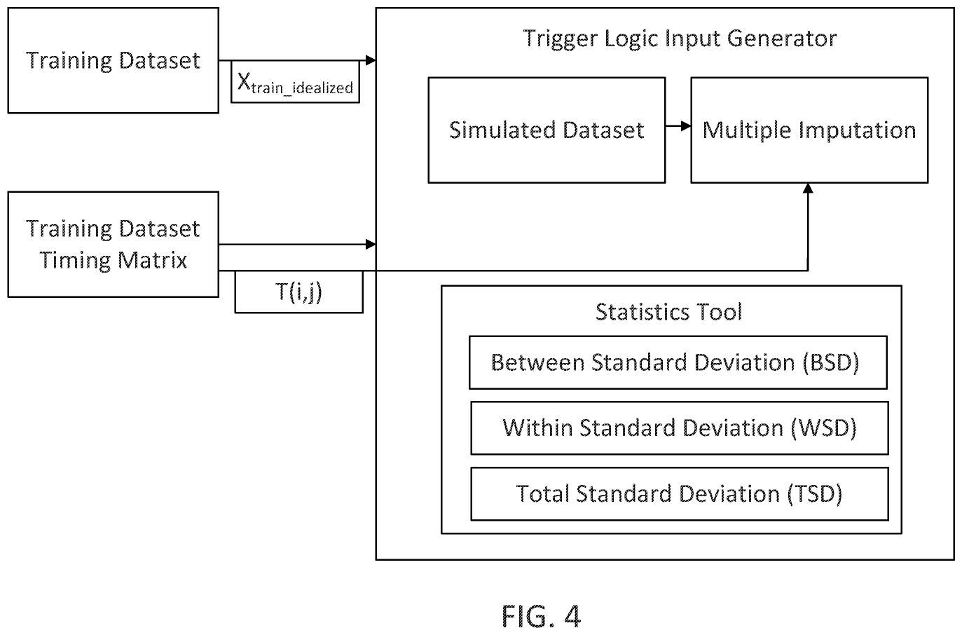

FIG. 4 illustrates a block diagram of a trigger logic input generator for a trigger system, in accordance with various embodiments. A training dataset timing matrix T(i,j), indicative of the times at which a feature j is available to a patient i, enables the construction of X_{train_idealized} in the trigger logic input generator. Accordingly, for each patient i and for each unique time in T(i,j), one can generate z instances per patient where z=|{T(i,)}|. Each instance corresponds to a specific time t in {T(i,)} and is a replicate of X_{train_idealized}(i,) unless T(i,j) is greater than t, in which case X_{train_idealized} (i,j) is replaced with NA. Based on M(Xᵢ(tₓ)), a statistics tool in the trigger logic input generator determines one or more metrices of a set of metrices including a “between standard deviation” value (BSD(M(Xᵢ(tₓ)))), a “within standard deviation” value (WSD(M(Xᵢ(tₓ)))), and a “total standard deviation” value (TSD(M(Xᵢ(tₓ)))).

In accordance to various embodiments, the trigger logic input generator includes a multiple imputation tool that creates m imputed instances, X_{i_m}(tₓ), for a given Xᵢ(tₓ), where X_{i_m} (tₓ) refer to the mₜₕ imputed instance of Xᵢ(tₓ). For each instance, X_{i_m}(tₓ), missing feature values are imputed with values drawn from a distribution defined by X_{train_idealized}. For example, in various embodiments, the multiple imputation tool may perform a multiple imputation by chained equations. For each imputed instance, M(X_{i_m}(tₓ)) is calculated using the modeling tool. The value BSD(Xi(tx)) is then defined as the standard deviation of the set of values {M(X_{i_1}(tₓ)), M(X_{i_2}(tₓ)), . . . , M(X_{i_m}(tₓ))))}. Accordingly, in various embodiments, the metric BSD(M(Xᵢ(tₓ))) may capture the variability induced in the outcome (e.g., medical outcome, financial outcome, and the like) by the missing data Yᵢ(tₓ).

The value for the metric WSD(M(Xᵢ(tₓ))) may include the inherent variability in a given prediction due to sampling from X_{train_idealized} and the variance of the response for a given input. Depending on the specific model used (e.g., logistic regression, random forests, SVM), estimates for the WSD(M(X_{i_m}(tₓ))) can be estimated using standard methods (e.g standard error of prediction interval, jackknife estimators, Bayesian estimators, maximum-likelihood based estimators, and the like).

The value for the metric TSD(M(Xᵢ(tₓ))) includes an estimate of the total variance for M(Xi (t)). In accordance with various embodiments, TSD may be obtained using the following mathematical expression:

$$\sqrt{\overline{WV} + ( 1 + \frac{1}{m} ) \cdot BV} \qquad ( 1 )$$

where W̅V̅ is the average within variance of (WV(M(X_{i_1}(tₓ))), WV(M(X_{i_2}(tₓ))), . . . , WV(M(X_{i_m}(tₓ)))) (where WV is defined as the square root of WSD) and BV is defined as the square root of BSD.

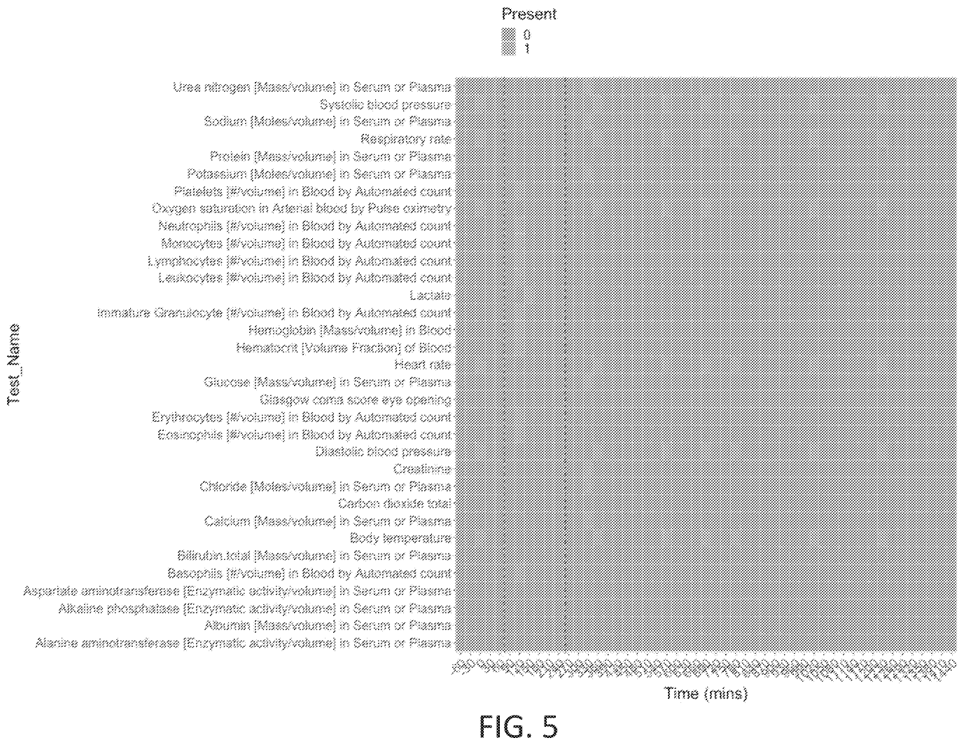

FIG. 5 illustrates an exemplary table of a dataset including a time sequence of multiple clinical tests, in accordance with various embodiments. The table indicates whether each of the multiple features (e.g., clinical data) are available or collected at a given collection time, for the patient. As can be seen from the table, multiple features may be collected at any given collection time period. Moreover, in accordance to various embodiments, the same clinical feature may be collected repeatedly, at different collection time periods (e.g., heart rate, respiratory rate, systolic blood pressure, body temperature, and others).

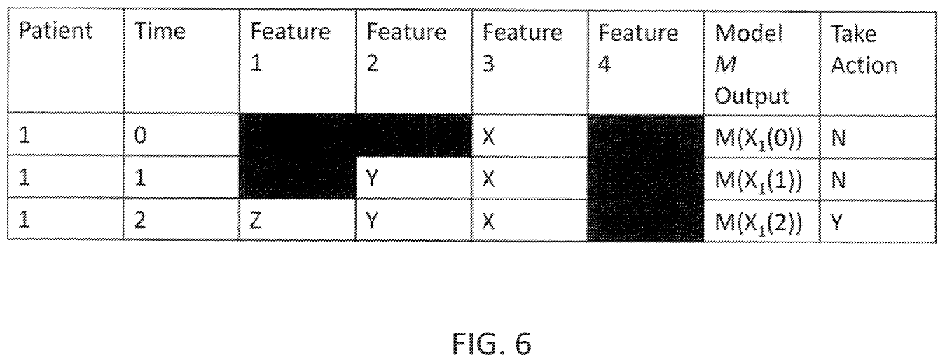

FIG. 6 is a table indicative of multiple features associated with a patient in a time sequence, and a trigger result for a healthcare action based on the features, in accordance with various embodiments. The table presents an in-depth look at a patient, demonstrating the arrival of certain data parameters and the moment when the trigger logic fires.

The table in FIG. 6 includes columns indicating: patient, time, feature(s), a model output M, and a decision result (e.g., ‘Take Action’, Y/N). Accordingly, for a first patient (e.g., patient 1), at time ‘0’, only Feature 3 has been collected ({X₁(0)}={NA, NA, X, NA}), and the model output M(X₁(0)) is indecisive, and so the system takes no action (N). At a subsequent time, 1, and for the same patient, a second feature is collected (e.g., Feature 2=Y, {X₁(1)}={NA, Y, X, NA}), and the model output M(X₁(1)) is still indecisive, and so the system takes no action (N), awaiting for further data to be collected. At a later time, 2, and for the same patient, a first feature is collected (e.g., Feature 1=Z, {X₁(1)}={Z, Y, X, NA}), and the model output M(X₁(2)) is then sufficient for the system to take action (Y), even though a fourth data may still be uncollected (e.g., Feature 4).

In accordance to various embodiments, the time entries in the table may occur at any given period of time, and the interval between the different time entries may or may not be the same, nor similar. In various embodiments, the interval between different time entries may be pre-selected, or random. Moreover, in various embodiments, more than one feature may be received at a given time interval. The table in FIG. 6 illustrates, according to various embodiments, how the trigger logic engine may be prepared to take an action even when there is one or more features missing in the input data. Accordingly, in various embodiments, the modeling engine may impute a value for the missing data, and based on statistical analysis of the model value and the imputed data, the trigger logic may determine to take an action with a pre-determined degree of certainty.

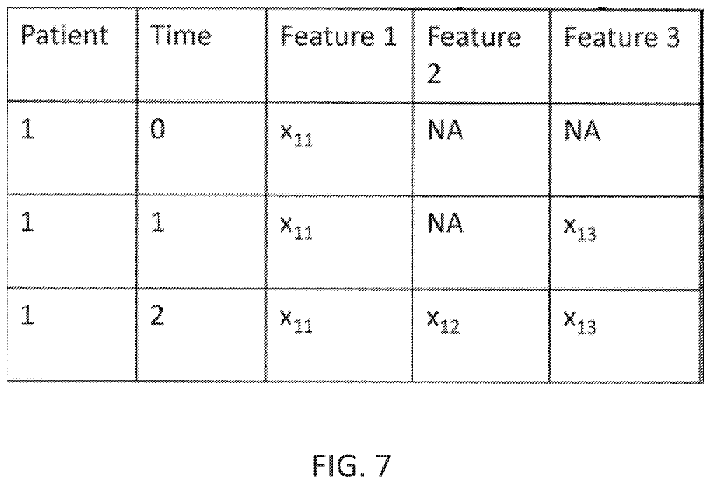

FIG. 7 is a partial illustration of an input table associated with features that may trigger an action for a patient in a time sequence, in accordance with various embodiments. The input table includes columns indicating: patient, time of entry, and feature(s). For simplicity, the table in FIG. 7 only illustrates three features and one patient, although it is understood that any number of features may be included, for one, two, or any number of patients. The input features are indicated as elements in a two-dimensional matrix, Xᵢⱼ, and the label NA indicates missing data. For example, element X₁₁ is the value of Feature 1 at times 0, 1 and 2, for patient 1. Element X₁₂ is the value of feature 2 at time 2, and element X₁₃ is the value of feature 3 at times 1 and 2.

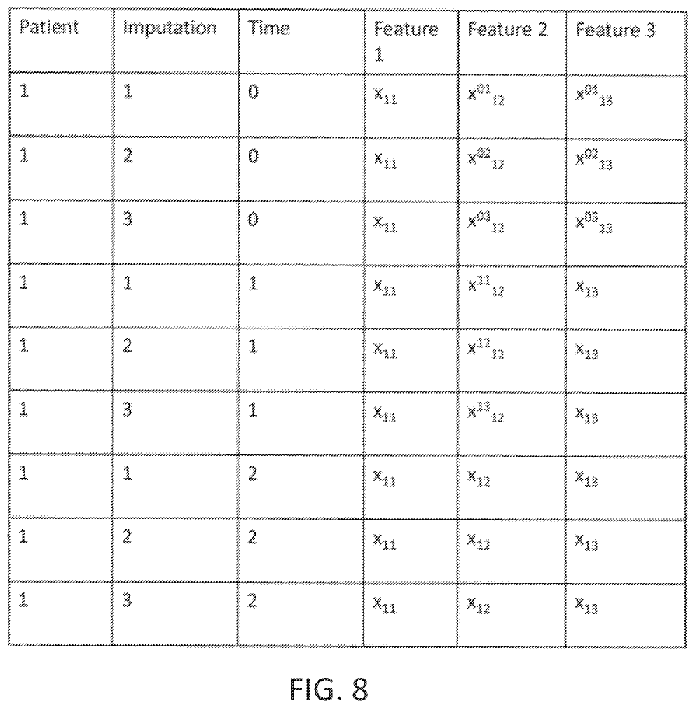

FIG. 8 is a partial illustration of a training dataset, in accordance with various embodiments. The training dataset in FIG. 8 includes an imputation column that lists missing data (e.g., data labeled ‘NA’ in FIG. 7) that are imputed by the modeling tool. According to various embodiments, the modeling tool may impute multiple values for a single feature at a given moment in time.

For example, at time ‘0’, Feature 2 and Feature 3 are missing in the original data (cf FIG. 7), and therefore three imputation rows (‘1’, ‘2’, and ‘3’) are included for each separate time value ‘0’, ‘1’, and ‘2’. For time ‘0’: in imputation ‘1’ the modeling tool imputes a value X⁰¹₁₂ to Feature 2, and a value X⁰¹₁₃ to Feature 3; in imputation ‘2’ the modeling tool imputes a value X⁰²₁₂ to Feature 2, and a value X⁰²₁₃ to Feature 3; and in imputation ‘3’ the modeling tool imputes a value X⁰³₁₂ to Feature 2, and a value X⁰³₁₃ to Feature 3. For time ‘1’: in imputation ‘1’ the modeling tool imputes a value X¹¹₁₂ to Feature 2; in imputation ‘2’ the modeling tool imputes a value X¹²₁₂ to Feature 2; and in imputation ‘3’ the modeling tool imputes a value X¹³₁₂ to Feature 2. Note that at time ‘1’, the modeling tool does not impute a value for Feature 3 because at that time, Feature 3 has collected a ‘true’ (or measured) value X₁₃. At time ‘2’, the modeling tool provides no imputed values because all three features have collected ‘true’ values X₁₁, X₁₂, and X₁₃.

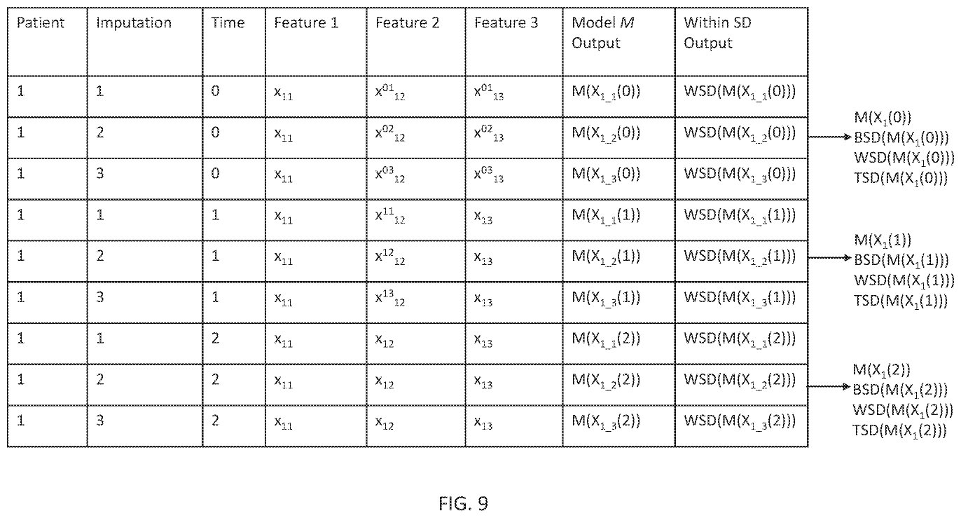

FIG. 9 is a partial illustration of a training dataset with model outputs and standard deviations, in accordance with various embodiments. Accordingly, the table in FIG. 9 is an extension of the table in FIG. 8, with the addition of a Model Output column, M(X(t)), and a within SD Output column WSD(M(X(t)))). The input data vector X(t) for the M and WSD columns varies according to the input data and the imputed data, and the time, t, is one of three time periods ‘0’, ‘1’, and ‘2’. For example, at time ‘0’, there are three model outputs, each associated with different data sets, containing different imputed data for Features 2 and 3: M(X_{1_1}(0)) for input data {X₁₁, X⁰¹₁₂, X⁰¹₁₃}; M(X_{1_2}(0)) for input data {X₁₁, X⁰²₁₂, X⁰²₁₃}; and M(X_{1_3}(0)) for input data {X₁₁, X⁰³₁₂, X⁰³₁₃}. Each of model outputs, M, may be associated to a different WSD given the data value for each feature, and the variance of the data values for each feature, whether the data values are collected from an instrument or device, manually entered by healthcare personnel, or imputed by the modeling tool. Accordingly, at a time ‘0’ there are three different WSD values: WSD(M(X_{1_1}(0))) for input data {X₁₁, X⁰¹₁₂, X⁰¹₁₃}; WSD(M(X_{1_2}(0))) for input data {X₁₁, X⁰²₁₂, X⁰²₁₃}; and WSD(M(X_{1_3}(0))) for input data {X₁₁, X⁰³₁₂, X⁰³₁₃}.

At time ‘1’, there are three model outputs, each associated with different data sets, containing different imputed data for Feature 2: M(X_{1_1}(1)) for input data {X₁₁, X¹¹₁₂, X₁₃}; M(X_{1_2}(1)) for input data {X₁₁, X¹²₁₂, X₁₃}; and M(X_{1_3}(1)) for input data {X₁₁, X¹³₁₂, X₁₃}. Each of model outputs, M, may be associated to three different WSD values: WSD(M(X_{1_1}(1))) for input data {X₁₁, X¹¹₁₂, X₁₃}; WSD(M(X_{1_2}(1))); for input data {X₁₁, X¹²₁₂, X₁₃}; and WSD(M(X_{1_3}(1))); and for input data {X₁₁, X¹³₁₂, X₁₃}.

At time ‘2’, there are three model outputs, M(X_{1_1}(2)) for input data {X₁₁, X₁₂, X₁₃}; M(X_{1_2}(2)) for input data {X₁₁, X₁₂, X₁₃}; and M(X_{1_3}(2)) for input data {X₁₁, X₁₂, X₁₃}. Each of model outputs, M, may be associated to three different WSD values: WSD(M(X_{1_1}(2))) for input data {X₁₁, X₁₂, X₁₃}; WSD(M(X_{1_2}(2))) for input data {X₁₁, X₁₂, X₁₃}; and WSD(M(X_{1_3}(2))) for input data {X₁₁, X₁₂, X₁₃}. Note that the values M(X_{1_1}(2)), M(X_{1_2}(2)) and M(X_{1_3}(2)) may be similar, because the input data {X₁₁, X₁₂, and X₁₃} is the same for the three model outputs. However, in some embodiments, the prior history of the model outputs for the different imputations at prior times may be different, and the modeling tool may provide different outputs for at least one of M(X_{1_1}(2)), M(X_{1_2}(2)), and M(X_{1_3}(2)).

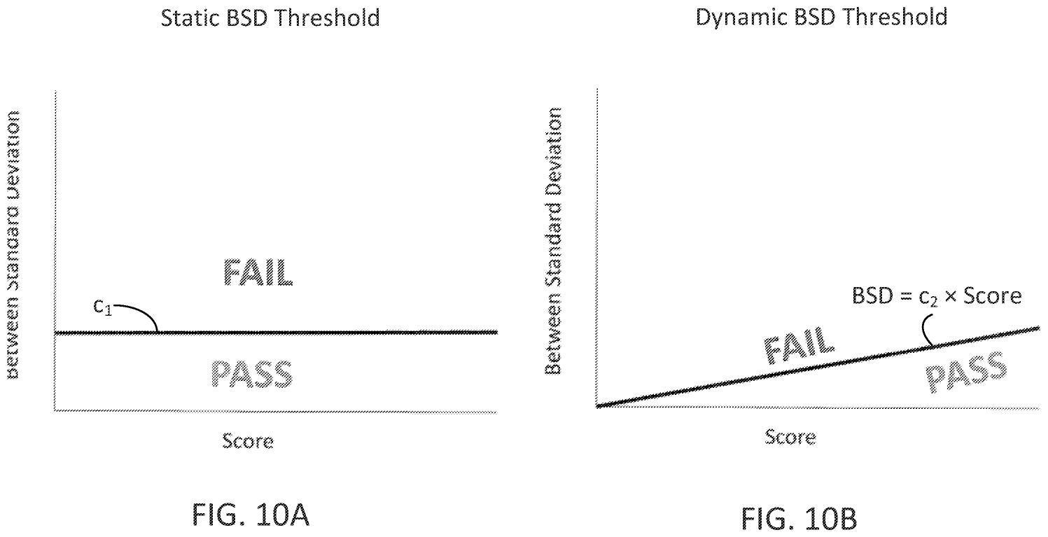

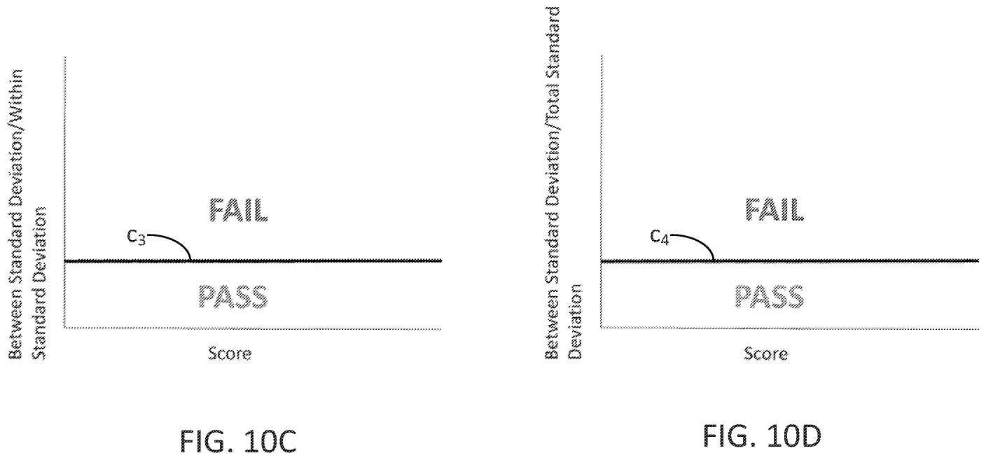

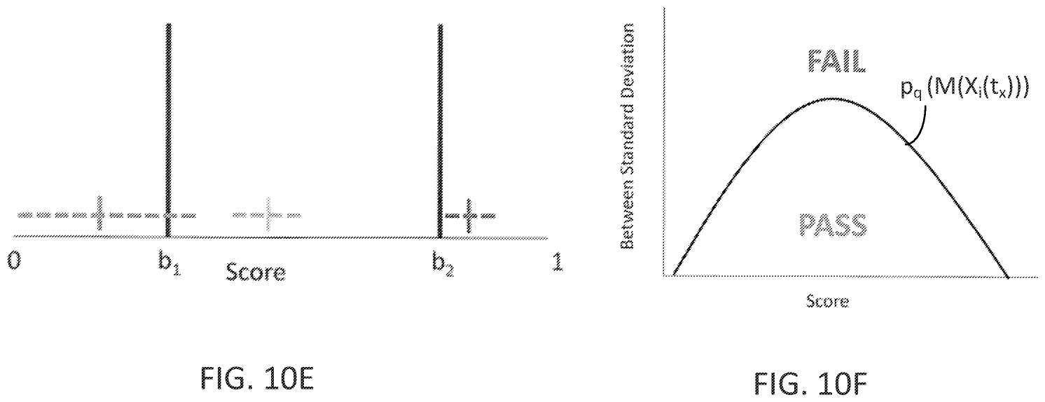

FIGS. 10A-10F are graphical illustrations of exemplary trigger logic rules, in accordance with various embodiments. For example, a stateless trigger logic rule may involve the trigger of an action based on the information available to the system at a given time, tₓ. Given M(Xᵢ(tₓ)), BSD(M(Xᵢ(tₓ))), WSD(M(Xᵢ(tₓ))), and TSD(M(Xᵢ (tₓ))), various rules can be employed that determine whether or not the system takes an action. The action taken by the system can be conditional on M(Xᵢ (tₓ)), BSD(M(Xᵢ (tₓ))), WSD(M(Xᵢ (tₓ))), and TSD(M(Xᵢ (tₓ))).

FIG. 10A illustrates an absolute BSD rule based on a static BSD threshold. Accordingly, when BSD(M(Xᵢ (tₓ))) is less than or equal to a pre-selected constant c₁, the system takes an action (“PASS”). Likewise, when BSD(M(Xᵢ(tₓ))) is greater than c₁, the system postpones the decision to time tₓ₊₁ (“FAIL”). Note that, in accordance with some embodiments, the absolute BSD rule may be independent of the specific value of the function M(Xᵢ(tₓ)) (also referred to hereinafter as ‘score’). More generally, a ‘score’ may be a function associated with the value of M(Xᵢ(tₓ)).

FIG. 10B illustrates a dynamic BSD threshold rule based on a ratio of BSD to the score. Accordingly, when the ratio BSD(M(Xᵢ (tₓ)))/M(Xᵢ (tₓ)) is less than or equal to a pre-selected constant c₂, the system takes an action (“PASS”). Likewise, when BSD(M(Xᵢ(tₓ))) is greater than c₂, the system postpones the decision to time tₓ₊₁ (“FAIL”).

FIG. 10C illustrates a logic rule based on a ratio of BSD to WSD. Accordingly, when BSD(M(Xᵢ(tₓ)))/WSD(Xᵢ(tₓ)) is less than or equal to a pre-selected constant c₃, the system takes an action (“PASS”). Likewise, when the ratio is greater than c₃, the system postpones the decision to time tₓ₊₁ (“FAIL”).

FIG. 10D illustrates a logic rule based on a ratio of BSD to TSD. Accordingly, when the ratio BSD(M(Xᵢ (tₓ)))/TSD(Xᵢ (tₓ)) is less than or equal to a pre-selected constant c₄, the system takes an action (“PASS”). Likewise, when the ratio is greater than c₄, the system postpones the decision to time tₓ₊₁ (“FAIL”).

FIG. 10E illustrates a logic rule based on a score boundary crossing. In accordance with various embodiments, scores may be discretized into risk categories (e.g., low, medium, high), separated by pre-selected boundaries, b₁, b₂, and the like. A method can be employed that takes into account the value of the score, the variance (between, within, or total) of the score, and the boundaries creating the risk categories (e.g., b₁, b₂). For example, the score M(Xᵢ(tₓ)) may be associated with or considered as a risk score indicating a level of risk for an undesirable outcome (e.g., clinical emergency, stock crash or bankruptcy, and the like). Accordingly, it may be desirable that the system takes action when a risk score greater than b1 or b2 is high, indicating a likelihood of an undesirable outcome.

In various embodiments, when the value of M(Xᵢ(tₓ)) is less than b₁, and the risk score and BSD satisfy the expression

M(Xᵢ(tₓ)))+c₅·BSD(M(Xᵢ(tₓ)))<b₁ (2)

(were c₅ is a pre-selected constant), then the system takes an action (“PASS”). Moreover, when the value of M(Xᵢ(tₓ)) is greater than b₁ and the risk score, M, and BSD satisfy the expression

M(Xᵢ(tₓ)))−c₅·BSD(M(Xᵢ(tₓ)))>b₁ (3)

then the system takes an action (“PASS”).

When the value of M(Xᵢ (tₓ)) is greater than b₂ and the risk score, M, and BSD satisfy the expression

M(Xᵢ(tₓ)))−c·BSD(M(Xᵢ(tₓ)))>b₂, (4)

then the system takes an action (“PASS”).

FIG. 10F illustrates a Polynomial Quantile Regression Boundary. First, a matrix B is created, where each row of B corresponds to BSD(M(Xᵢ (t_{f}))) for a given patient i at a fixed time t_{f} for some or all patients. In various embodiments, t_{f} is relative to some common event experienced by most or all patients such that t_{f} is standardized. Given the matrix, B, in various embodiments, a polynomial quantile regression is performed on B for a given quantile q, creating a function p_{q}. For a given M(Xᵢ (tₓ)), the system postpones an action for at least time tₓ₊₁ (“FAIL”) when BSD(M(Xᵢ (tₓ))) is greater than or equal to p_{q}(M(Xᵢ (tₓ))). Likewise, the system takes an action when BSD is less than p_{q} (“PASS”).

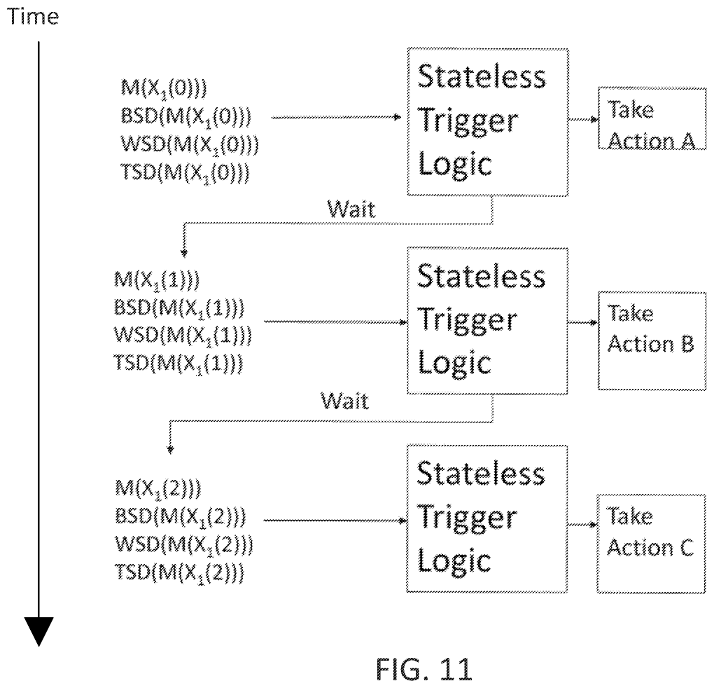

FIG. 11 illustrates a time sequence of actions triggered by a trigger logic engine with a stateless trigger logic rule, in accordance with various embodiments. Based on an input data Xi (tₓ), the trigger logic input generator determines M(Xᵢ (tₓ)), BSD(M(Xᵢ (tₓ))), WSD(M(Xᵢ (tₓ))), TSD(M(Xᵢ (tₓ))). Further, the trigger logic input generator feeds the inputs to the trigger logic engine to use with stateless trigger logic rules R (cf. FIGS. 10A-10F). Accordingly, a trigger logic engine may include a function R(M(Xᵢ (tₓ)), BSD(M(Xᵢ (tₓ))), WSD(M(Xᵢ (tₓ))), TSD(M(Xᵢ (tₓ))) that generates an output ‘0’ to postpone an action (“FAIL”) or ‘1’, to trigger an action (“PASS”).

In accordance to various embodiments, a database coupled with the trigger logic engine stores the values M(Xᵢ (t_{trigger})) and t_{trigger} in a matrix X_{R_simulated_stateless} for a given stateless trigger logic rule R and for each patient i. The value t_{trigger} may include a time in {T(i, j)} (e.g., the least time, or one of the lower time values in the set) such that R(M(Xᵢ (tₓ)), BSD(M(Xᵢ (tₓ))), WSD(M(Xᵢ (tₓ))), TSD(M(Xᵢ (tₓ)))=1. In various embodiments, the database also includes standard diagnostic metrics and prognostic metrics for X_{R_simulated_stateless}. In various embodiments, the database may also store metrics associated with the time distribution of the trigger and the percentage of patients for which the system triggers (e.g., R=1).

As illustrated in FIG. 11, at different times tₓ=0, 1 and 2, under a stateless trigger logic rule, different actions are taken by the system (action A, action B, and action C, respectively), independently of one another.

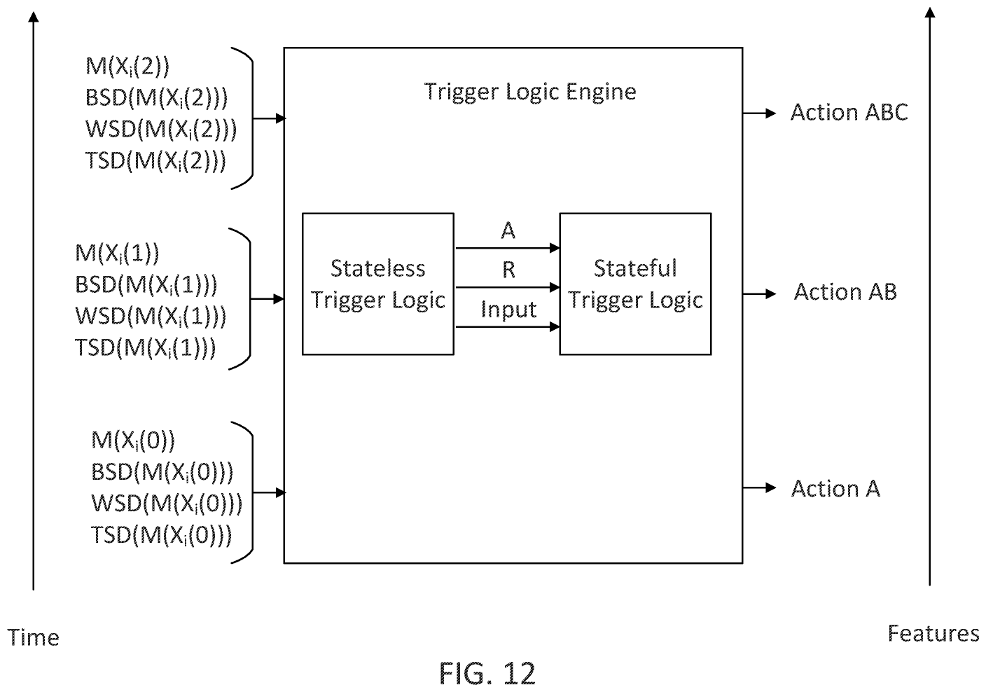

FIG. 12 illustrates a time sequence of actions triggered by a trigger logic engine with a stateful trigger logic, in accordance with various embodiments. A stateful trigger logic may include state-dependent logic rules wherein input data collected in previous times, ty, is considered for a decision at a given time, tₓ, with y<x. In various embodiments, the trigger logic engine is communicably coupled with a database storing a matrix, X_{R_simulated_stateful}, that includes values M(Xᵢ(t_{m_trigger})) and t_{m_trigger} in X_{R_simulated_stateful} where t_{m_trigger} refers to the mₜₕ time such that R(M(Xᵢ (tₓ)), BSD(M(Xᵢ (tₓ))), WSD(M(Xᵢ (tₓ))), TSD(M(Xᵢ (tₓ)))=1 (e.g., the m, time when the system was triggered for a given patient). The value of m can be dependent on current (tₓ) and prior states of a patient based on state dependent trigger logic. The database may also store standard diagnostic and prognostic metrics for X_{R_simulated_stateful}. In various embodiments, the database may also include metrics regarding the time distribution of the trigger and the percentage of patients for which the system triggers (e.g., R=1). Such configuration may be desirable to increase accuracy of the prognostics in a less restrictive time constraint environment.

In applications with a greater tolerance for time, the trigger logic may be implemented in a state dependent manner. For instance, in a stateless environment, the output of the trigger logic engine can be represented as R(M(Xi (tx)), BSD(M(Xi (tx))), WSD(M(Xi (tx))), TSD(M(Xi (tx))), where R refers to a stateless trigger logic rule that outputs a binary number indicating to trigger (1) or not trigger (0). Further, a function, A, may be defined to specify the action that the system may take to prevent an undesirable outcome, or to produce a desirable outcome (e.g., administering a medication, providing a medical procedure, investing or divesting funds, and the like). Accordingly, A may be represented as a function, A(M(Xi (tx)), BSD(M(Xi (tx))), WSD(M(Xi (tx))), TSD(M(Xi (tx))). In a state dependent environment, R and A can be functions not only of M(Xi (tx)), BSD(M(Xi (tx))), WSD(M(Xi (tx))), and TSD(M(Xi (tx)) but also of M(Xi (ty)), BSD(M(Xi (ty))), WSD(M(Xi (ty))), TSD(M(Xi (ty)) for any y<x. The conditional logic governing this may be arbitrarily complex.

Accordingly, in various embodiments, the trigger logic engine including a stateful logic engine produces actions A, AB, and ABC at different times tₓ=0, 1 and 2. Action AB may be a result not only of the values {M(Xi (0)), BSD(M(Xi (0))), WSD(M(Xi (0))), TSD(M(Xi (0))}, but also of the values {M(Xi (1)), BSD(M(Xi (1))), WSD(M(Xi (1))), TSD(M(Xi (1))}. Likewise, action ABC may be the result of the values {M(Xi (0)), BSD(M(Xi (0))), WSD(M(Xi (0))), TSD(M(Xi (0))} at time tₓ=0, the values {M(Xi (1)), BSD(M(Xi (1))), WSD(M(Xi (1))), TSD(M(Xi (1))} at time tₓ=1, and the values {M(Xi (2)), BSD(M(Xi (2))), WSD(M(Xi (2))), TSD(M(Xi (2))} at time tₓ=2.

In various embodiments, matrices X_{R_simulated_stateless} and X_{R_simulated_stateful} can be used to quantify the influence of a given set of features conditional on prior features available in the trigger logic engine. In various embodiments, the trigger logic engine is configured to select a set of features that mostly influenced a decision for a given action, A, for each entry in either X_{R_simulated_stateless} and X_{R_simulated_stateful}. For example, in various embodiments, the trigger logic engine may identify the values of Xᵢ(t_{trigger}) and t_{trigger}, or the values of Xᵢ (t_{m_trigger}) and t_{m_trigger} that have more relevance in the outcome of the function A.

In various embodiments, the trigger logic engine may identify the feature values that arrive prior to t_{trigger} in Xᵢ(t_{trigger}) or prior to t_{m_trigger} in Xᵢ(t_{m_trigger}) in matrices X_{R_simulated_stateless} and X_{R_simulated_stateful} to determine the set of features driving a given action, A. In various embodiments, the trigger logic engine accesses the data structure in the matrix T(i,j) (which may be stored in the database) to make this determination. Accordingly, the trigger logic engine may provide a matrix D_{conditional} wherein each row corresponds to t_{trigger} or t_{m_trigger} and to the name of the corresponding set of features, F, that instigated t_{trigger} or t_{m_trigger}. In some embodiments, matrix D_{conditional} includes, more coarsely, the class, C, or set of features driving a given action. The class, C, may include vital features such as, CBC features, CMP features, financial features, seasonal features, and the like. The matrix D_{conditional} may be stored in the database, for use by the trigger logic engine as desired.

In various embodiments, the trigger logic engine may also determine a percentage of entries of F or C in matrix D_{conditional}. Accordingly, the percentage of entries for F and C in D_{conditional} may be used in the modeling tool to assess the conditional influence of the features F, or classes of features, C, in the trigger logic engine. In various embodiments, a conditional influence of a feature Fₖ or class Cₖ is given in relation to one or more of the features or classes of features: e.g., the influence of Fk given Fx, Fy, . . . , Fz, or the influence of Ck given Cx, Cy, . . . , Cz. In various embodiments, features Fx, Fy, . . . , Fz and classes of features Cx, Cy, . . . , Cz may vary for each patient.

In various embodiments, the trigger logic engine may determine the isolated effect of Fₖ or Cₖ, in driving a given action, A. Accordingly, the trigger logic engine may generate matrices X_{R_simulated_stateless} and X_{R_simulated_stateful} wherein columns for each row of T are permuted. For example, a matrix T_{permuted} is formed by independent shuffling of the columns in timing matrix T(i,j) for all i in T. Using Tₚₑᵣₘᵤₜₑ, the trigger logic engine generates X_{R_simulated_stateless} and X_{R_simulated_stateful}, and it also generates D_{isolated}, similarly to D_{conditional}. Accordingly, the trigger logic engine may determine the isolated influence from the percentage presence of the feature Fₖ or class Cₖ in the matrix D_{isolated}.

More generally, various embodiments may include a trigger logic engine that determines the conditional effect of any arbitrary feature Fk or class Ck given Fx, Fy, . . . , Fz or Ck given Cx, Cy, . . . , Cz, where Fx, Fy, . . . , Fz and Cx, Cy, . . . , Cz are the same for most or all patients. This can be accomplished by appropriately permuting each T(i,) for all i in T such that a particular relationship holds, e.g., Fk arrives after Fx, Fy, . . . , Fz, for most or all patients.

In various embodiments, a state dependent logic in a trigger logic engine may identify when a score triggers again (e.g., R=1) within T minutes of the initial trigger. More specifically, in various embodiments, the time T after initial trigger (R=1) may be set to 90 minutes. Action A may be presenting to the physician that the patient is currently in the low-risk category, meaning they are unlikely to benefit from prompt administration of antibiotics, and action B may be presenting to the physician that the patient is currently in the medium-risk category, meaning they are likely to moderately benefit from prompt administration of antibiotics with regard to relevant clinical outcomes.

When the current model value, M, indicates a medium-risk category (in which the action to be taken by the system is B) but was previously in the low-risk category (in which the action taken by the system was A, where A is distinct from B), then the trigger logic engine may trigger the system to perform B (e.g., AB=B predicated on the occurrence of A). In various embodiments, action B itself may be dependent on A. Likewise, action ABC may indicate that action C is taken, predicated that actions A and B have been taken (in that order).

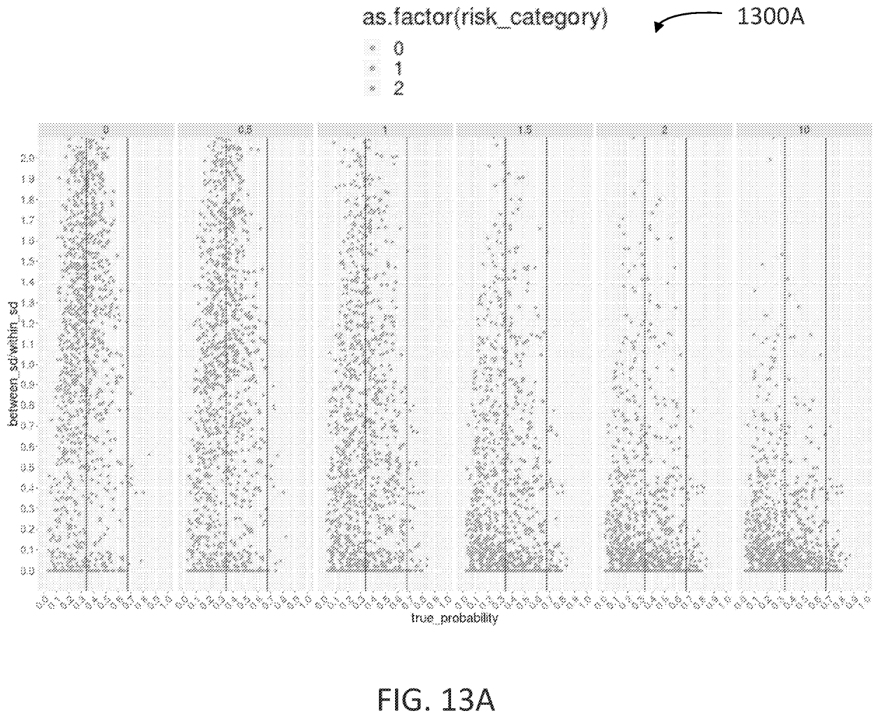

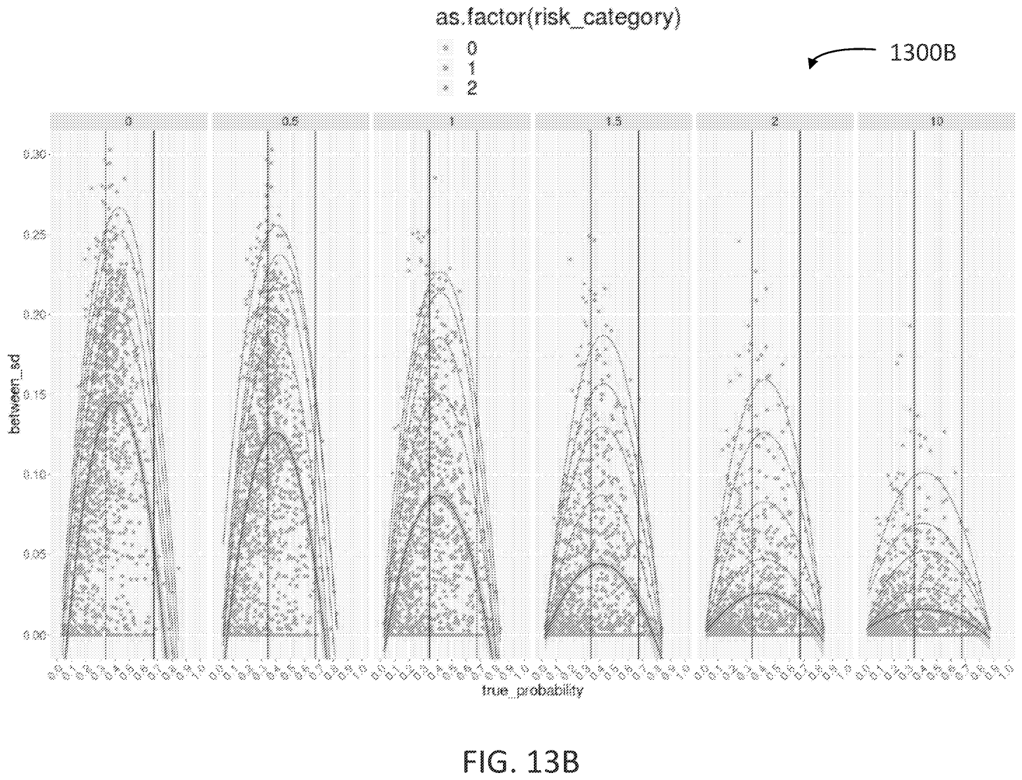

FIGS. 13A-13B are charts 1300A and 1300B (hereinafter, collectively referred to as “charts 1300”) illustrating a time evolution of a standard deviation distribution over a risk factor, measured for multiple patients, over six different time intervals (listed as time, in hours). The abscissae (X-axis) in charts 1300 indicate the risk factor. The ordinates (Y-axis) indicate a BSD/WSD ratio in chart 1300A (cf. FIG. 13A) and a BSD value in chart 1300B. Each facet in the plot refers to a particular time in hours relative to a fixed time point.

Charts 1300 are exemplary illustrations of a trigger logic engine designed in the context of sepsis, a disease defined as life-threatening organ dysfunction caused by a dysregulated host response to an infection. Early therapy—particularly using empiric antibiotics—leads to improved outcomes. However, vague presenting symptoms make the recognition of sepsis difficult and leads to increased mortality. The initial recognition and treatment of sepsis often occurs in the emergency department (ED) setting, which can be chaotic and understaffed, complicating the ability of medical providers to reliably identify and treat this syndrome. Various embodiments resolve this problem with modeling tools as disclosed herein, to assess the likelihood that a patient is septic and to assess the severity of their state.

In various embodiments, modeling tools and trigger logic engines as disclosed herein utilize features routinely measured for patients suspected of sepsis. Some of these features may be present in the electronic medical record (EMR) for the patient (e.g., vitals, CBC, count associated laboratory results, CMP, and the like), and also utilize parameters specifically measured for hospitalized patients suspected of sepsis that may not be present in the electronic medical record (e.g., novel plasma proteins, nucleic acids, and the like). Accordingly, a trigger logic engine trained for sepsis diagnostic and treatment may operate in a highly time-sensitive environment, in which streaming data arrives from different sources quickly and asynchronously.

In various embodiments, the modeling tool includes a function, M, indicative of a risk score, e.g., ranging from 0 to 1. The risk score may be categorized within three ranges as either: low, medium, or high risk. The trigger logic engine may be an action function, A, including outcomes such as presenting the risk score to a physician, nurse, and/or relevant healthcare personnel, or postponing a decision to a later time (e.g., by a selected period of time, or when a new symptom or medical feature appears, and the like). Action function, A, may depend on the risk factor and also on other stateful information.

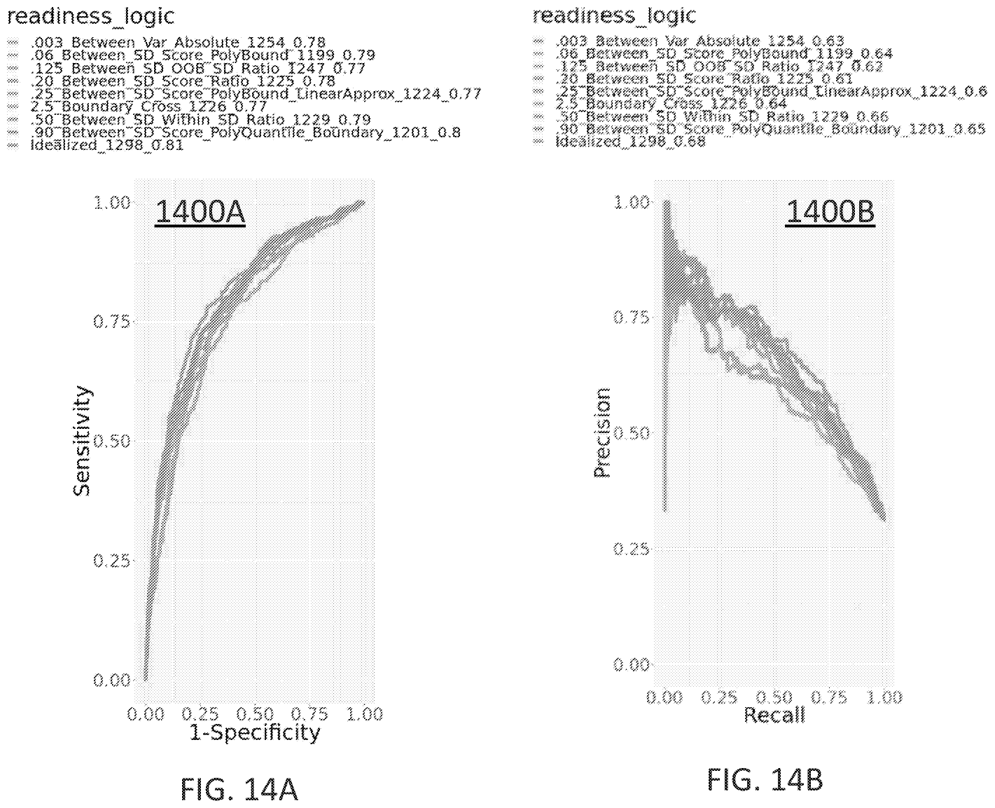

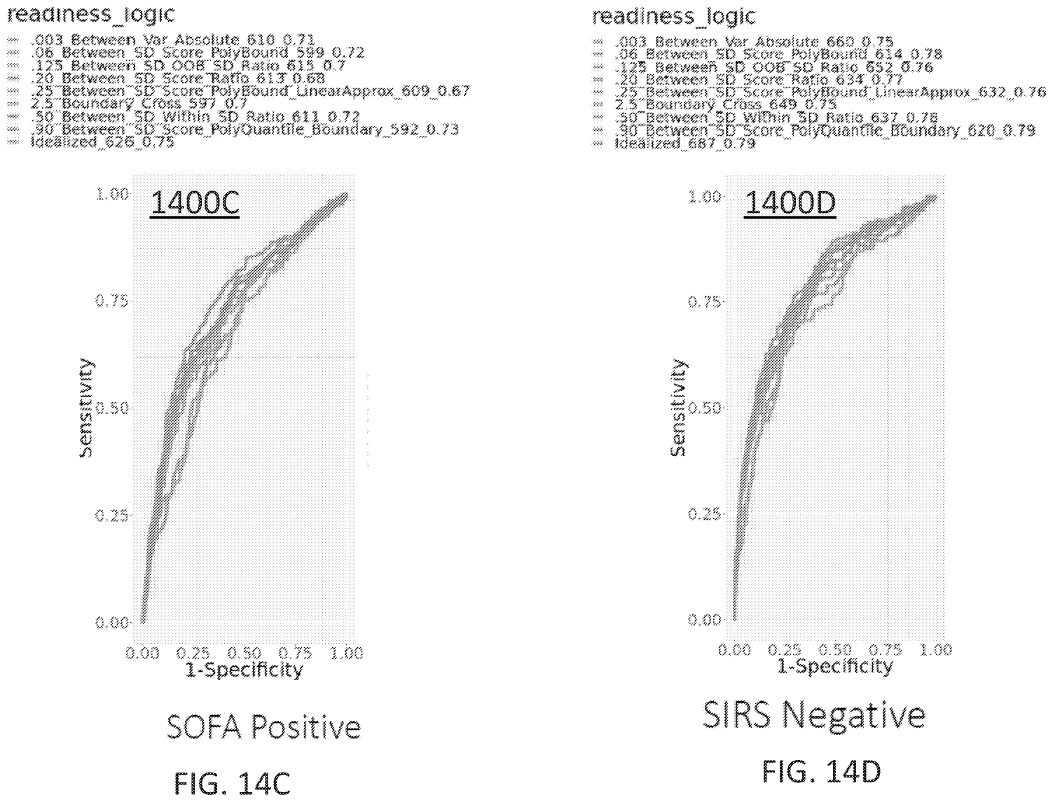

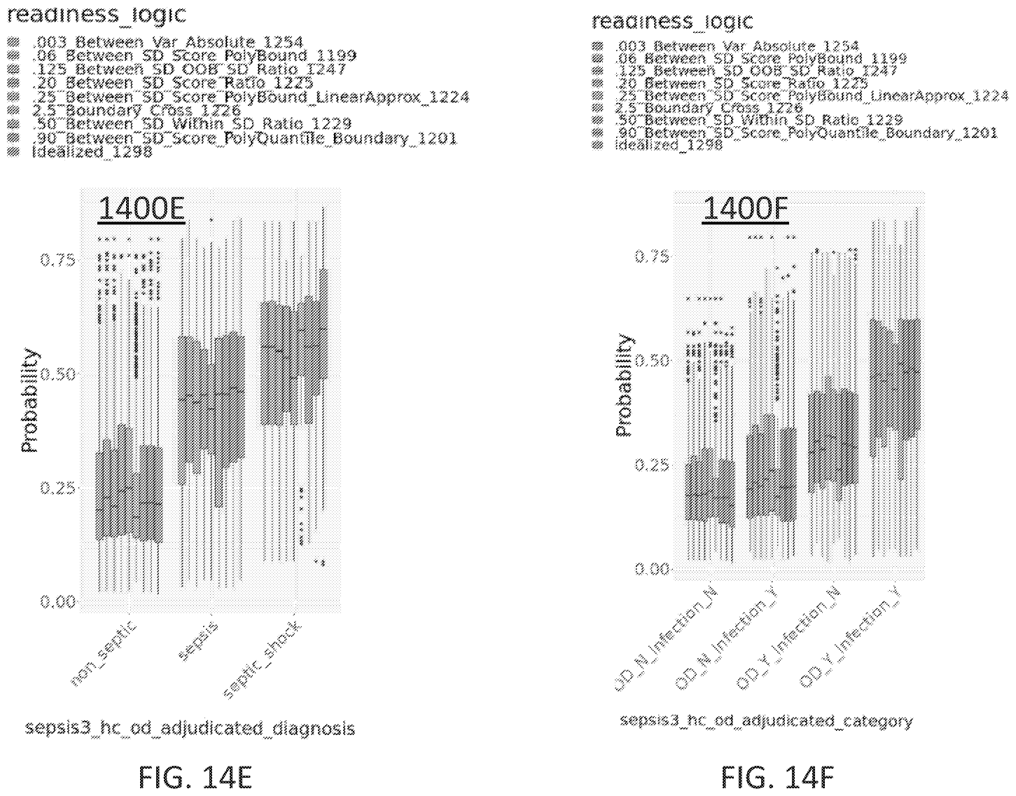

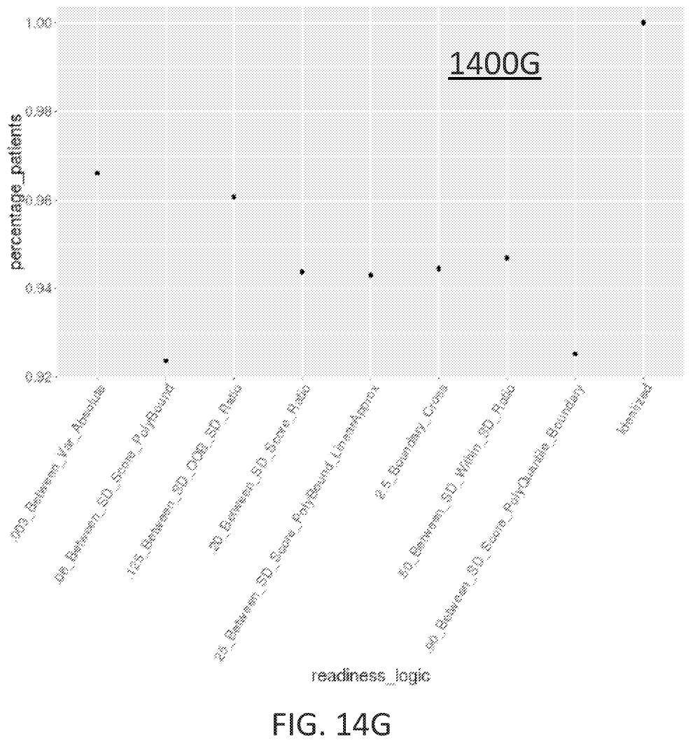

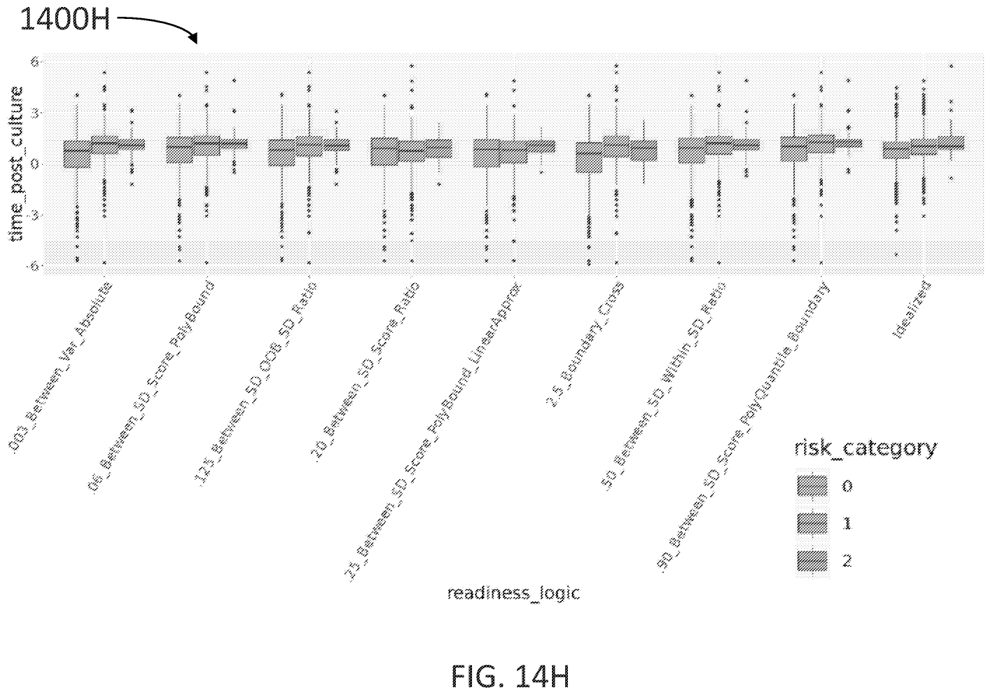

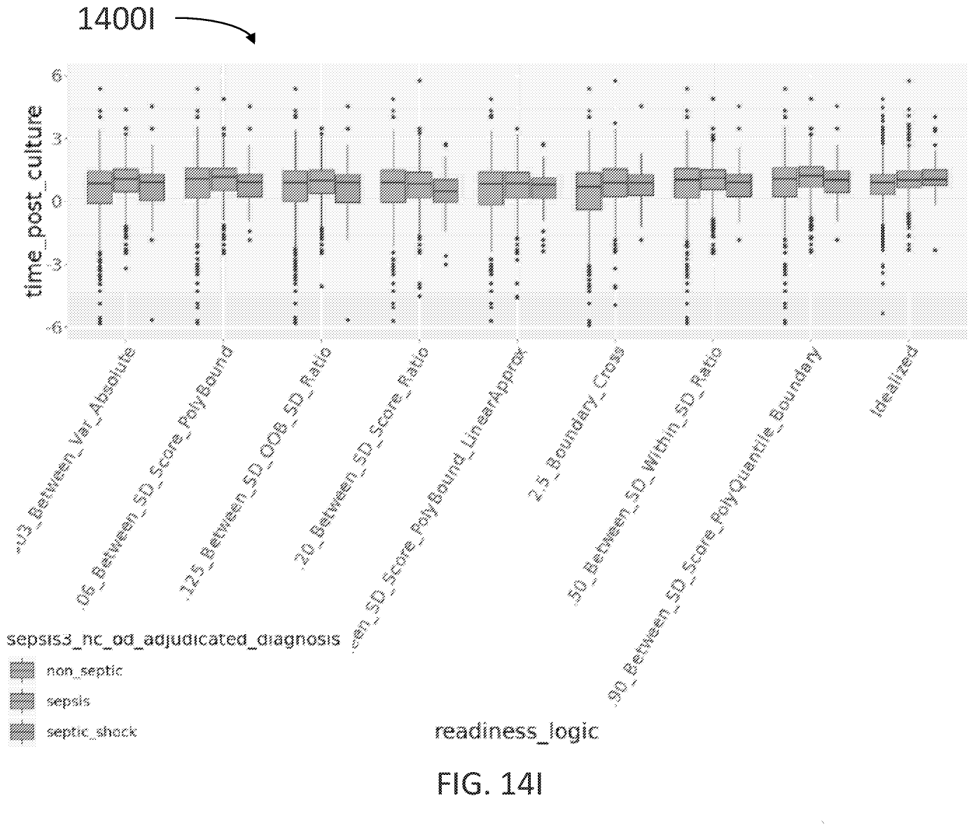

FIGS. 14A-14I are charts 1400A-I (hereinafter, collectively referred to as “charts 1400”) illustrating a diagnostic performance with a stateless trigger logic engine, in accordance with various embodiments. According to various embodiments, charts 1400 may be obtained with a statistics tool in a trigger logic engine, cooperating with a modeling tool and an imputation tool (cf. trigger logic engine 240, modeling tool 242, statistics tool 244, and imputation tool 246). Accordingly, the statistics tool may provide standard deviation (e.g., BSD, WSD, and TSD) and variance values for input data and for imputed data using one or more mathematical expressions as disclosed herein (cf Eq. 1). Charts 1400 are collected in various exemplary case scenarios in a stateless configuration (wherein the modeling tool considers the latest information available to make imputations on missing data), for illustrative purposes only. Each color in the charts refers to the diagnostic performance of a specific stateless trigger logic rule R. Without limitation, various embodiments may include a 0.003 between variance absolute value imputation tool; a 0.6 BSD combined with a polynomial boundary for the score; a 0.125 ratio of BSD to OOB SD; a 0.2 BSD to score ratio; a 2.5 boundary cross; a 0.9 BSD combined with a polynomial quantile boundary for the score. ‘Idealized’ refers to the scenario where one waits for all available data before providing an output (which is optimal for accuracy but suboptimal in terms of providing timely predictions).

FIG. 14A is a chart 1400A illustrating a sensitivity v. specificity response of a trigger logic engine, according to various embodiments.

FIG. 14B is a chart 1400B illustrating a precision v. recall performance of a trigger logic engine, according to various embodiments.

FIG. 14C is a chart 1400C illustrating a sensitivity v. specificity response of a trigger logic engine, according to various embodiments. Chart 1400C applies to a sequential organ failure assessment (SOFA) positive score.

FIG. 14D is a chart 1400D illustrating a sensitivity v. specificity response of a trigger logic engine, according to various embodiments. Chart 1400D applies to a systemic inflammatory response syndrome (SIRS) negative analysis.

FIG. 14E is a chart 1400E illustrating a probability spread of a sepsis adjudicated diagnosis in various embodiments, using a trigger logic engine consistent with the present disclosure. Three different conditions are illustrated: non-septic, sepsis, and septic shock.

FIG. 14F is a chart 1400F illustrating a probability spread for a sepsis adjudicated category in various embodiments, using a trigger logic engine consistent with the present disclosure. Four different categories are indicated: OD_N_infection_N, OD_N_infection_Y, OD_Y_infection_N, and OD_Y_infection_Y.

FIG. 14G is a chart 1400G indicating a percentage of patients impacted by decisions made based on a trigger logic engine as disclosed herein, for the various embodiments listed above. The lowest impact is found for a 0.06 BSD combined with a polynomial boundary for the score, at a slightly over 92% impact. The largest impact is found for decisions made for a 0.003 between variance absolute at an almost 97% impact.

FIG. 14H is a chart 1400H indicating a timing to a decision made by a trigger logic engine as disclosed herein, for the various embodiments disclosed above. The time axis (vertical axis, or ordinates) indicate a time to decision in arbitrary units. The output of the trigger logic engine in chart 1400H indicates one of three risk categories for a sepsis diagnostic (‘0’, ‘1’, and ‘2’). In general, the variance spread of the risk category seems to be higher for the low-risk data, and lower for the high-risk data.

FIG. 14I is a chart 1400I indicating a timing to a decision made by a trigger logic engine as disclosed herein, for the various embodiments disclosed above. The time axis (vertical axis, or ordinates) indicate a time to decision in arbitrary units. The decision for the trigger logic engine in chart 1400I is to adjudicate a sepsis diagnosis according to three conditions, ‘non-septic’, ‘sepsis’, and ‘septic shock’. In general, the variance spread of the risk category seems to be higher for the low-risk data, and lower for the high-risk data.

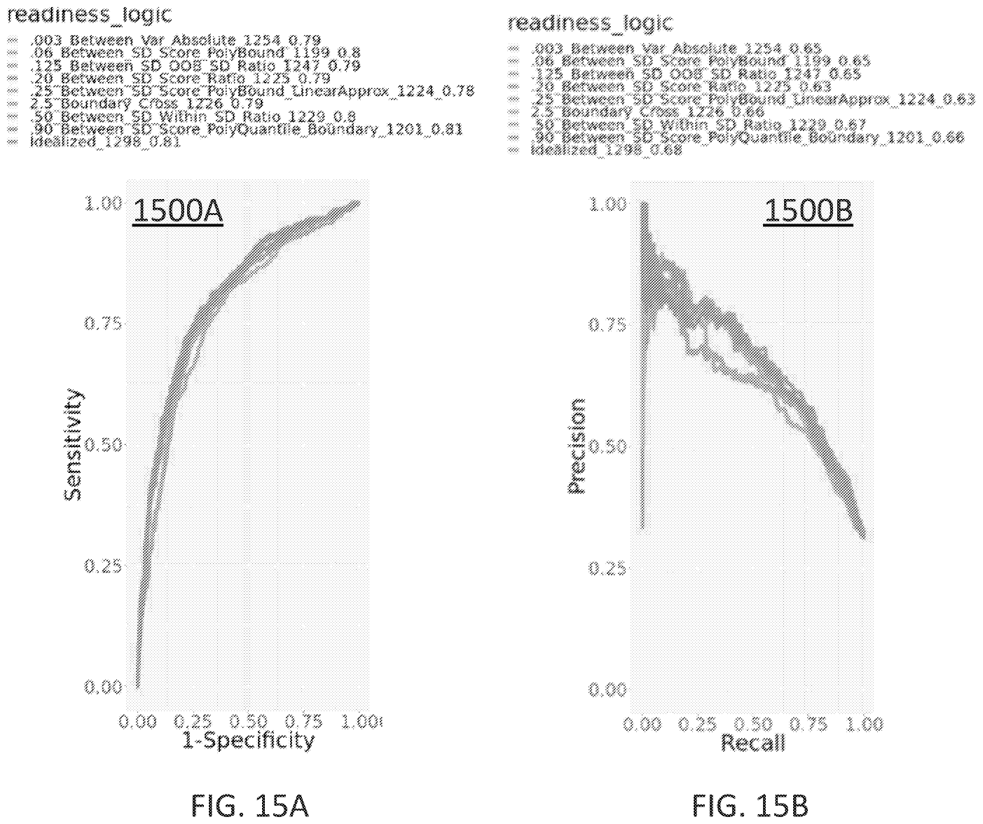

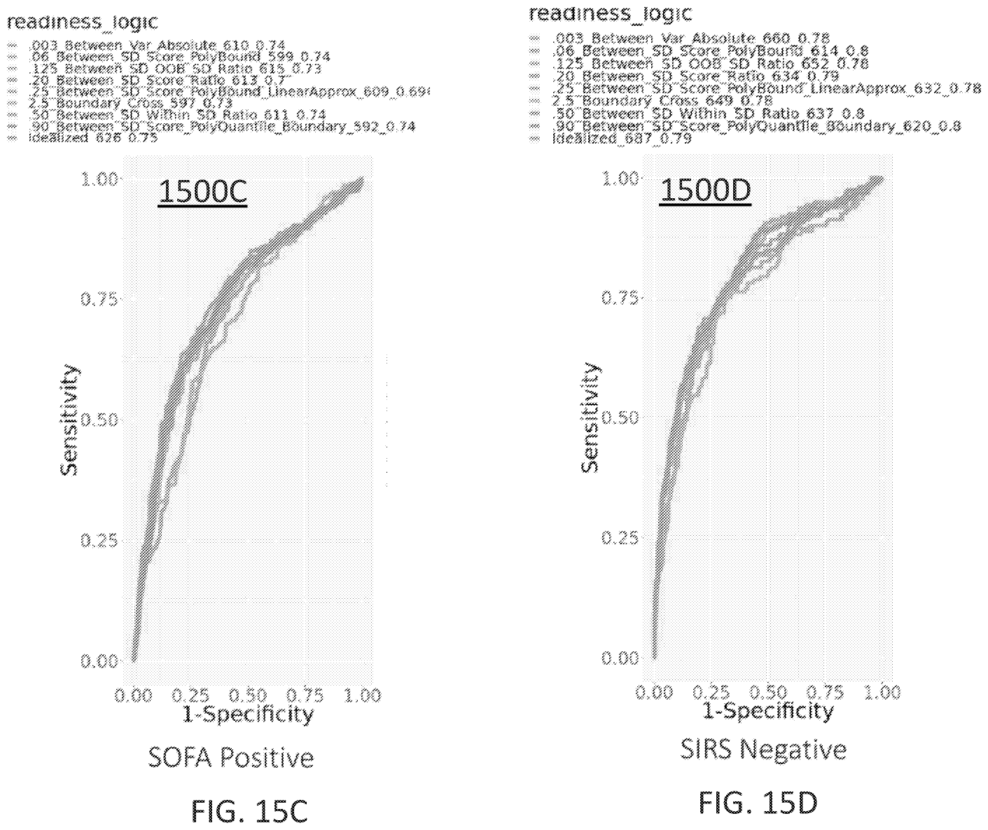

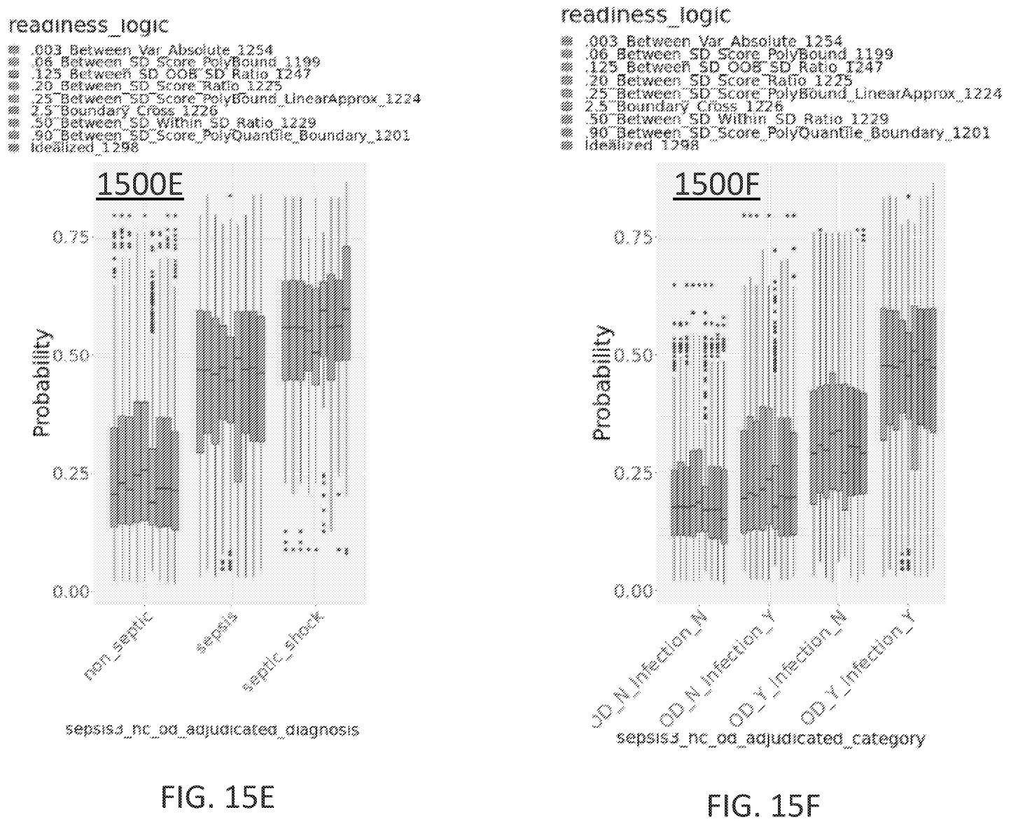

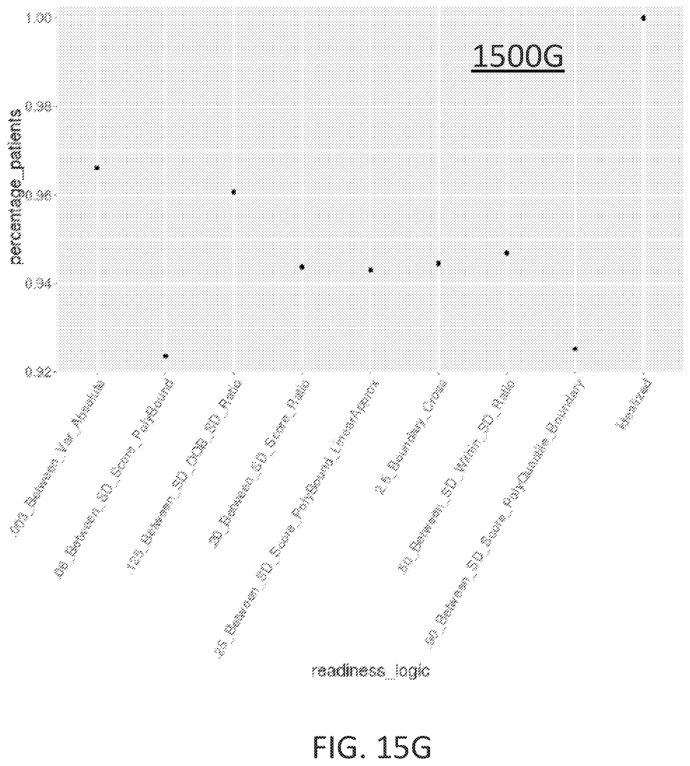

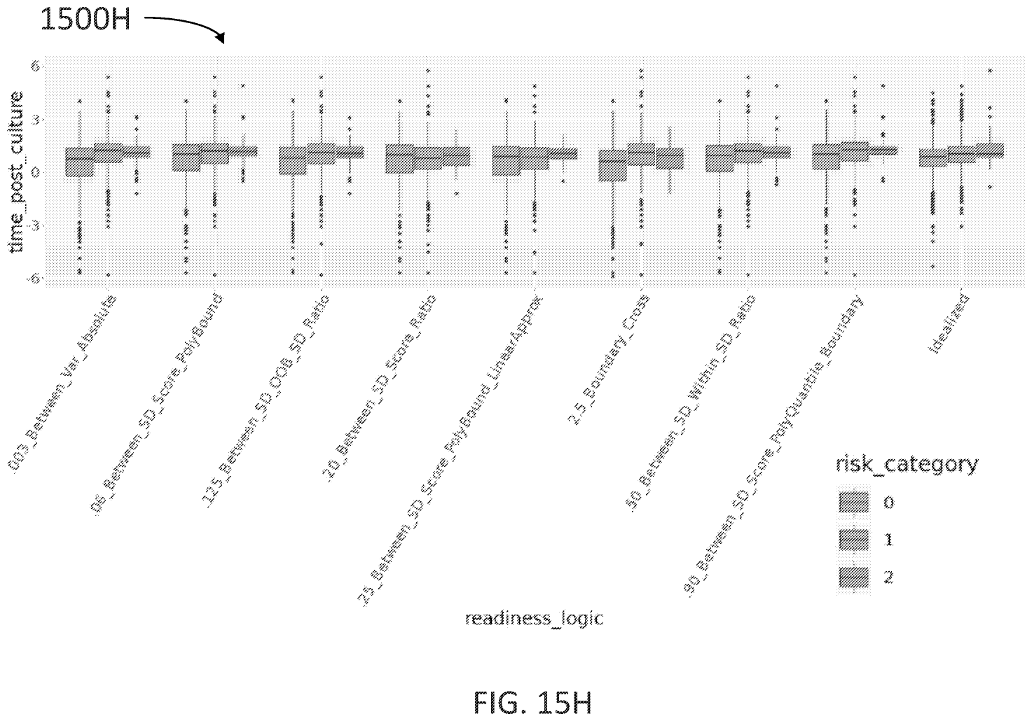

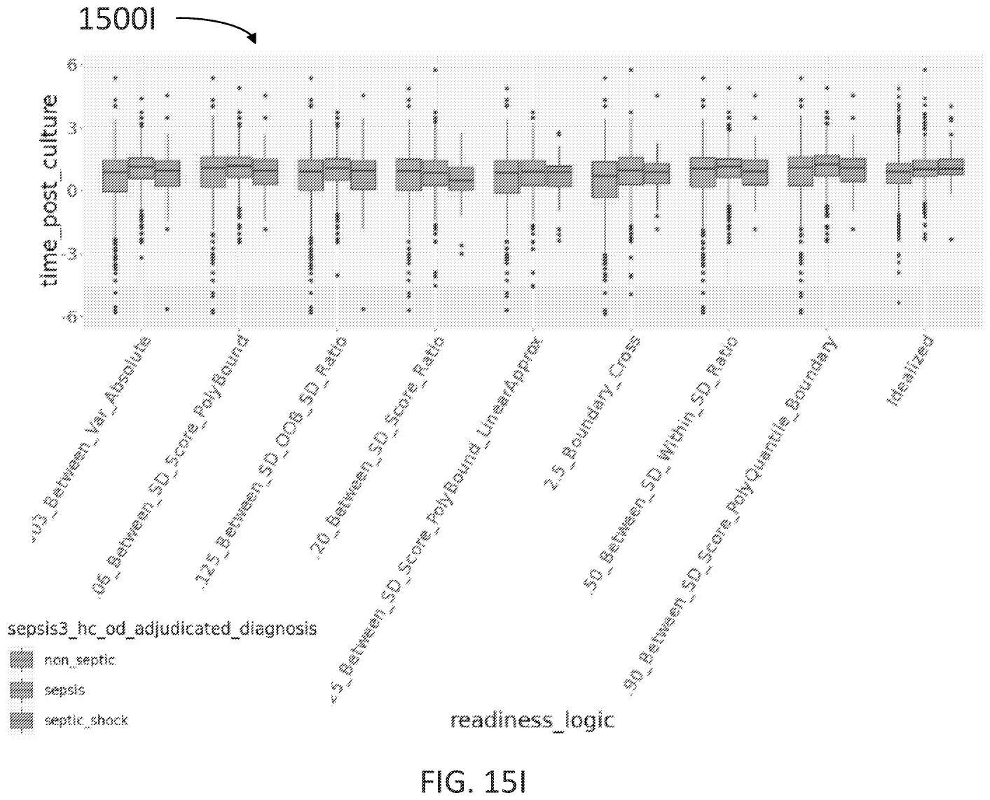

FIGS. 15A-15I are charts (1500A-I, hereinafter, collectively referred to as ‘charts 1500’) illustrating a diagnostic performance with a stateful trigger logic engine, according to various embodiments. According to various embodiments, charts 1500 may be obtained with a statistics tool in a trigger logic engine, cooperating with a modeling tool and an imputation tool (cf. trigger logic engine 240, modeling tool 242, statistics tool 244, and imputation tool 246). Accordingly, the statistics tool may provide standard deviation (e.g., BSD, WSD, and TSD) and variance values for input data and for imputed data using one or more mathematical expressions as disclosed herein (cf Eq. 1). Charts 1500 are collected in various exemplary case scenarios in a stateful configuration (wherein the modeling tool considers previously collected and/or imputed information in addition to the latest information available to make imputations on missing data), for illustrative purposes only. Each color in the charts refers to the diagnostic performance of a specific stateless trigger logic rule R wrapped around a stateful condition. The specific stateful condition used in this case was if the score triggers again within T minutes of the initial trigger and the score is currently in the medium-risk category (in which the action to be taken by the system is M) but was previously in the low-risk category (in which the action taken by the system was L, where L is distinct from M), then trigger the system to perform M. Note that M itself may be dependent on L. Without limitation, various embodiments may include a 0.003 between variance absolute value imputation tool; a 0.6 BSD combined with a polynomial boundary for the score; a 0,125 ratio of BSD to OOB SD; a 0.2 BSD to score ratio; a 2.5 boundary cross; a 0.9 BSD combined with a polynomial quantile boundary for the score. ‘Idealized’ refers to the scenario where one waits for all available data before providing an output (which is optimal for accuracy but suboptimal in terms of providing timely predictions).

FIG. 15A is a chart 1500A illustrating a sensitivity v. specificity response of a trigger logic engine, according to various embodiments.

FIG. 15B is a chart 1500B illustrating a precision v. recall performance of a trigger logic engine, according to various embodiments.

FIG. 15C is a chart 1500C illustrating a sensitivity v. specificity response of a trigger logic engine, according to various embodiments. Chart 1500C applies to a sequential organ failure assessment (SOFA) positive score.

FIG. 15D is a chart 1500D illustrating a sensitivity v. specificity response of a trigger logic engine, according to various embodiments. Chart 1500D applies to a systemic inflammatory response syndrome (SIRS) negative analysis.

FIG. 15E is a chart 1500E illustrating a probability spread of a sepsis adjudicated diagnosis in various embodiments, using a trigger logic engine consistent with the present disclosure. Three different conditions are illustrated: non-septic, sepsis, and septic shock.

FIG. 15F is a chart 1500F illustrating a probability spread for a sepsis adjudicated category in various embodiments, using a trigger logic engine consistent with the present disclosure. Four different categories are indicated: OD_N_infection_N, OD_N_infection_Y, OD_Y_infection_N, and OD_Y_infection_Y.

FIG. 15G is a chart 1500G indicating a percentage of patients impacted by decisions made based on a trigger logic engine as disclosed herein, for the various embodiments listed above. The lowest impact is found for a 0.06 BSD combined with a polynomial boundary for the score, at a slightly over 92% impact. The largest impact is found for decisions made for a 0.003 between variance absolute at an almost 97% impact.

FIG. 15H is a chart 1500H indicating a timing to a decision made by a trigger logic engine as disclosed herein, for the various embodiments disclosed above. The time axis (vertical axis, or ordinates) indicate a time to decision in arbitrary units. The output of the trigger logic engine in chart 1500H indicates one of three risk categories for a sepsis diagnostic (‘0’, ‘1’, and ‘2’). In general, the variance spread of the risk category seems to be higher for the low-risk data, and lower for the high-risk data.

FIG. 15I is a chart 1500I indicating a timing to a decision made by a trigger logic engine as disclosed herein, for the various embodiments disclosed above. The time axis (vertical axis, or ordinates) indicate a time to decision in arbitrary units. The decision for the trigger logic engine in chart 1500I is to adjudicate a sepsis diagnosis according to three conditions, ‘non-septic’, ‘sepsis’, and ‘septic shock’. In general, the variance spread of the risk category seems to be higher for the low-risk data, and lower for the high-risk data.

As expected, the timing to a decision in charts 1500H and 1500I is slightly higher for the stateful logic configuration in the trigger logic engine, as compared to the stateless logic configuration (cf. charts 1400H and 1400I).

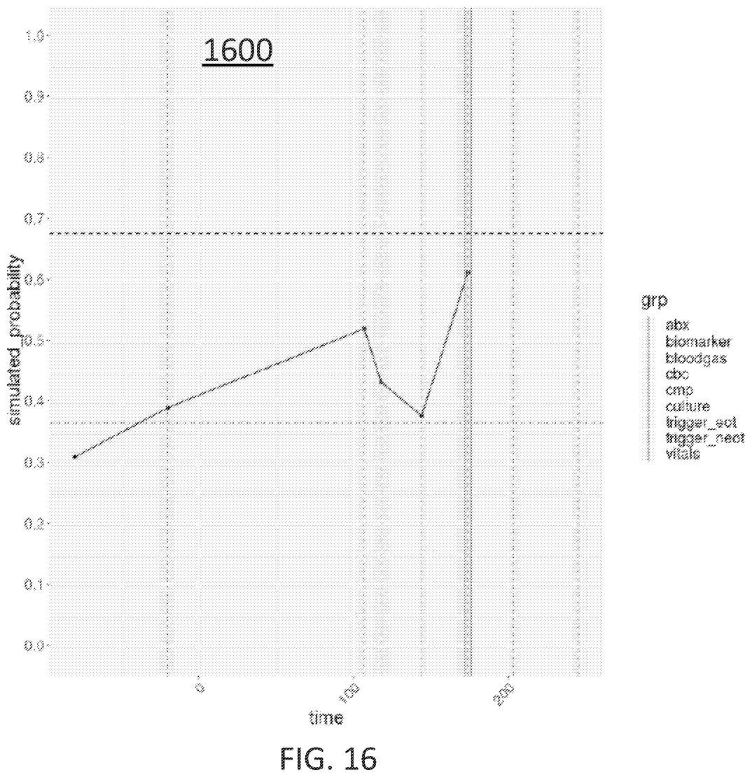

FIG. 16 is a chart 1600 for illustrating a probability to take action for a patient over time based on multiple medical features, in accordance with various embodiments.

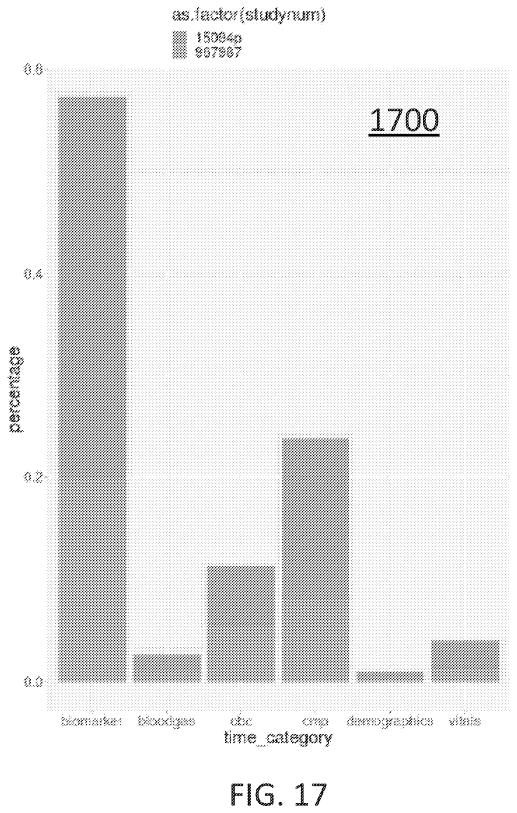

FIG. 17 is a bar plot 1700 of a risk factor for two different sets of patients over several medical features, in accordance with various embodiments. Bar plot 1700 is a visualization of the results of D_{conditional}, and illustrates a time-sensitive trigger using X_{R_simulated_stateless}. Accordingly, each row in the X_{R_simulated_stateless} data matrix for bar plot 1700 corresponds to t_{trigger} and the name of the corresponding set or class of clinical data (e.g., vitals, CBC, CMP, and the like) that instigated t_{trigger}. Bar plot 1700 illustrates an exemplary percentage of the conditional influence on the trigger logic engine of a specific clinical data entry for two different groups of patients, each from separate clinical sites.

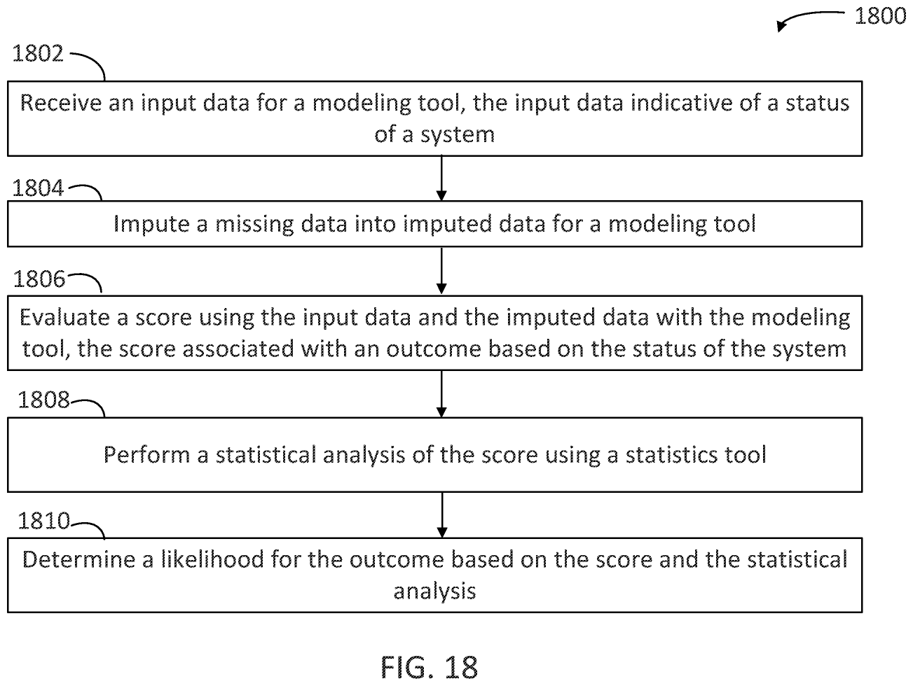

FIG. 18 is a flow chart illustrating steps in a method 1800 to perform a medical action on a patient based on multiple medical features received or imputed over a time sequence, in accordance with various embodiments. Method 1800 may be performed at least partially by any one of client devices coupled to one or more servers through a network (e.g., any one of servers 130 and any one of client devices 110, and network 150). For example, in accordance to various embodiments, the servers may host one or more medical devices or portable computer devices carried by medical or healthcare personnel. Client devices 110 may be handled by a user such as a worker or other personnel in a healthcare facility, or a paramedic in an ambulance carrying a patient to the emergency room of a healthcare facility or hospital, an ambulance, or attending to a patient at a private residence or in a public location remote to the healthcare facility. At least some of the steps in method 1800 may be performed by a computer having a processor executing commands stored in a memory of the computer (e.g., processors 212 and memories 220). In accordance to various embodiments, the user may activate an application in the client device to access, through the network, a trigger logic engine in the server (e.g., application 222 and trigger logic engine 240). The trigger logic engine may include a modeling tool, a statistics tool, and an imputation tool to retrieve, supply, and process clinical data in real-time, and provide an action recommendation thereof (e.g., modeling tool 242, statistics tool 244, and imputation tool 246). Further, steps as disclosed in method 1800 may include retrieving, editing, and/or storing files in a database that is part of, or is communicably coupled to, the computer, using, inter-alia, a trigger logic engine (e.g., databases 252). Methods consistent with the present disclosure may include at least some, but not all, of the steps illustrated in method 1800, performed in a different sequence. Furthermore, methods consistent with the present disclosure may include at least two or more steps as in method 1800 performed overlapping in time, or almost simultaneously.

Step 1802 includes receiving an input data for a modeling tool, the input data indicative of a status of a system.

Step 1804 includes imputing a missing data into imputed data for the modeling tool. In various embodiments, step 1804 includes applying a multiple imputation technique to generate N copies of the patient's data for a specific instance of a patient's data at a certain time. In various embodiments, step 1804 may include replacing the missing data value with one imputed data value. In some embodiments, step 1804 may include replacing each missing data value with one or more imputed data values, to evaluate the variability in the imputation model. For example, step 1804 may include creating ‘N’ imputed data values for each missing data value, wherein each imputed data value is predicted from a slightly different model in the modeling tool, to reflect sampling variability.

Step 1806 includes evaluating a score using the input data and the imputed data with the modeling tool, the score associated with an outcome based on the status of the system. For each copy of the data, step 1806 may include providing the input data (including the imputed data) into the modeling tool and generating a prediction of the outcome.

Step 1808 includes performing a statistical analysis of the score using a statistics tool. In various embodiments, step 1808 includes generating estimates for the BSD, the WSD, and the TSD.

Step 1810 includes determining a likelihood for the outcome based on the score and the statistical analysis. In various embodiments, step 1810 may include applying conditional logic to the BSD, the WSD, the TSD, the score, and other outputs, when the modeling tool provides the score. For example, in various embodiments, step 1810 may include applying a condition when the BSD is less than a pre-selected value, then trigger a specific output or action. In some embodiments, step 1810 may include postponing a decision or an output until a further time, when the conditional logic is false, or not satisfied.

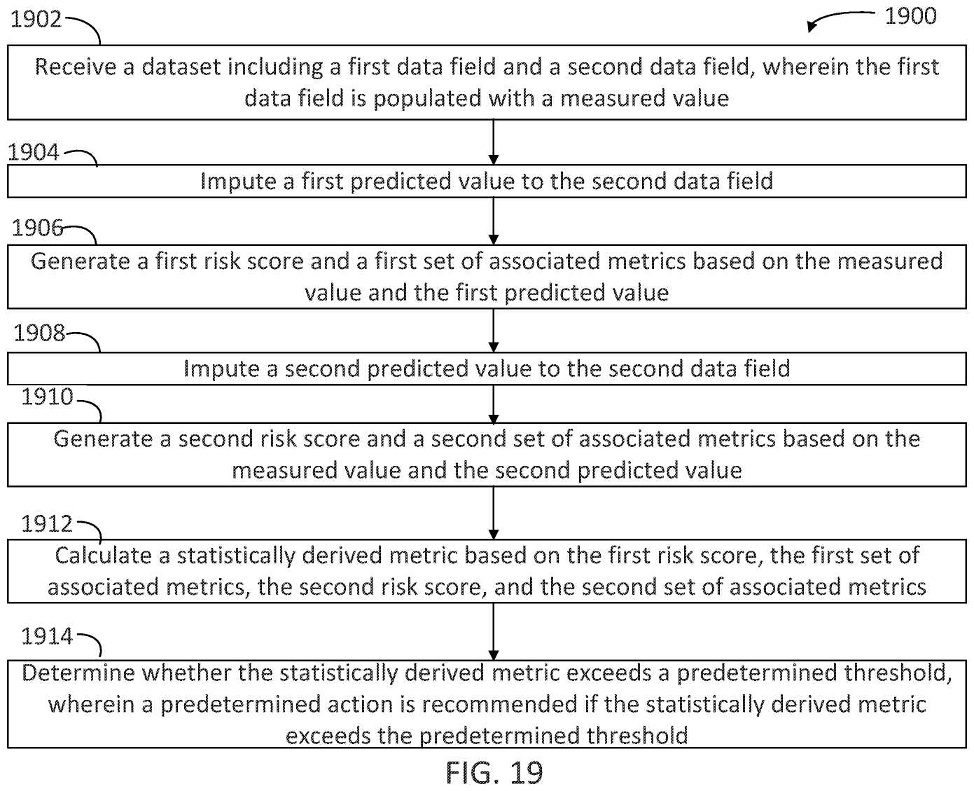

FIG. 19 is a flow chart illustrating steps in a method 1900 to perform a medical action on a patient based on multiple medical features received or imputed over a time sequence, in accordance with various embodiments. Method 1900 may be performed at least partially by any one of client devices coupled to one or more servers through a network (e.g., any one of servers 130 and any one of client devices 110, and network 150). For example, in accordance to various embodiments, the servers may host one or more medical devices or portable computer devices carried by medical or healthcare personnel. The client devices may be handled by a user such as a worker or other personnel in a healthcare facility, or a paramedic in an ambulance carrying a patient to the emergency room of a healthcare facility or hospital, an ambulance, or attending to a patient at a private residence or in a public location remote to the healthcare facility. At least some of the steps in method 1900 may be performed by a computer having a processor executing commands stored in a memory of the computer (e.g., processors 212 and memories 220). In accordance to various embodiments, the user may activate an application in the client device to access, through the network, a trigger logic engine in the server (e.g., application 222 and trigger logic engine 240). The trigger logic engine may include a modeling tool, a statistics tool, and an imputation tool to retrieve, supply, and process clinical data in real-time, and provide an action recommendation thereof (e.g., modeling tool 242, statistics tool 244, and imputation tool 246). Further, steps as disclosed in method 1900 may include retrieving, editing, and/or storing files in a database that is part of, or is communicably coupled to, the computer, using, inter-alia, a trigger logic engine (e.g., databases 252). Methods consistent with the present disclosure may include at least some, but not all, of the steps illustrated in method 1900, performed in a different sequence. Furthermore, methods consistent with the present disclosure may include at least two or more steps as in method 1900 performed overlapping in time, or almost simultaneously.

Step 1902 includes receiving a dataset including a first data field and a second data field, wherein the first data field is populated with a measured value. In accordance to various embodiments, step 1902 may include receiving, in a server, the measured value from a client device, through a network.

Step 1904 includes imputing a first predicted value to the second data field. In accordance to various embodiments, step 1904 further includes determining the first predicted value based on the measured value and a conditional rule relating the first data field to the second data field. In accordance to various embodiments, step 1904 includes determining the first predicted value using a model in a trigger logic engine.

Step 1906 includes generating a first risk score and a first set of associated metrics based on the measured value and the first predicted value. In accordance to various embodiments, step 1906 includes determining a variability induced in the first risk score by the first predicted value in a between standard deviation value. In accordance to various embodiments, step 1906 includes determining a variability induced in the first risk score by a sampling variability in a within standard deviation. In accordance to various embodiments, step 1906 includes determining a total standard deviation that includes a between standard deviation and a within standard deviation.

Step 1908 includes imputing a second predicted value to the second data field.

Step 1910 includes generating a second risk score and a second set of associated metrics based on the measured value and the second predicted value.

Step 1912 includes calculating a statistically derived metric based on the first risk score, the first set of associated metrics, the second risk score, and the second set of associated metrics. In accordance to various embodiments, step 1912 includes determining a ratio between a first standard deviation value and a second standard deviation value, each of the first standard deviation value and the second standard deviation value selected from the first set of associated metrics or from the second set of associated metrics. In accordance to various embodiments, step 1912 includes calculating a polynomial function of the first risk score or the second risk score and comparing a standard deviation selected from the first set of associated metrics and the second set of associated metrics to the polynomial function.

Step 1914 includes determining whether the statistically derived metric exceeds a predetermined threshold, wherein a predetermined action is recommended when the statistically derived metric exceeds the predetermined threshold. In accordance to various embodiments, the first set of associated metrics corresponds to a first collection time, the second set of associated metrics corresponds to a second collection time, and step 1914 includes using a stateful logic after the first collection time and the second collection time. In accordance to various embodiments, the first set of associated metrics corresponds to a first collection time, the second set of associated metrics corresponds to a second collection time, and step 1914 includes using a stateless logic after one of the first collection time or the second collection time. In accordance to various embodiments, the dataset includes clinical data for a patient, the clinical data having one of a complete blood count, a comprehensive metabolic panel, or a blood gas; and step 1914 includes determining a confidence level for a likelihood that the patient will suffer a septic shock. In accordance to various embodiments, step 1914 includes selecting the predetermined action based on a previous dataset including a first previous value for the first data field and a second previous value for the second data field. In accordance to various embodiments, step 1914 may further include providing a graphic chart for a display, the graphic chart illustrating the statistically derived metric.

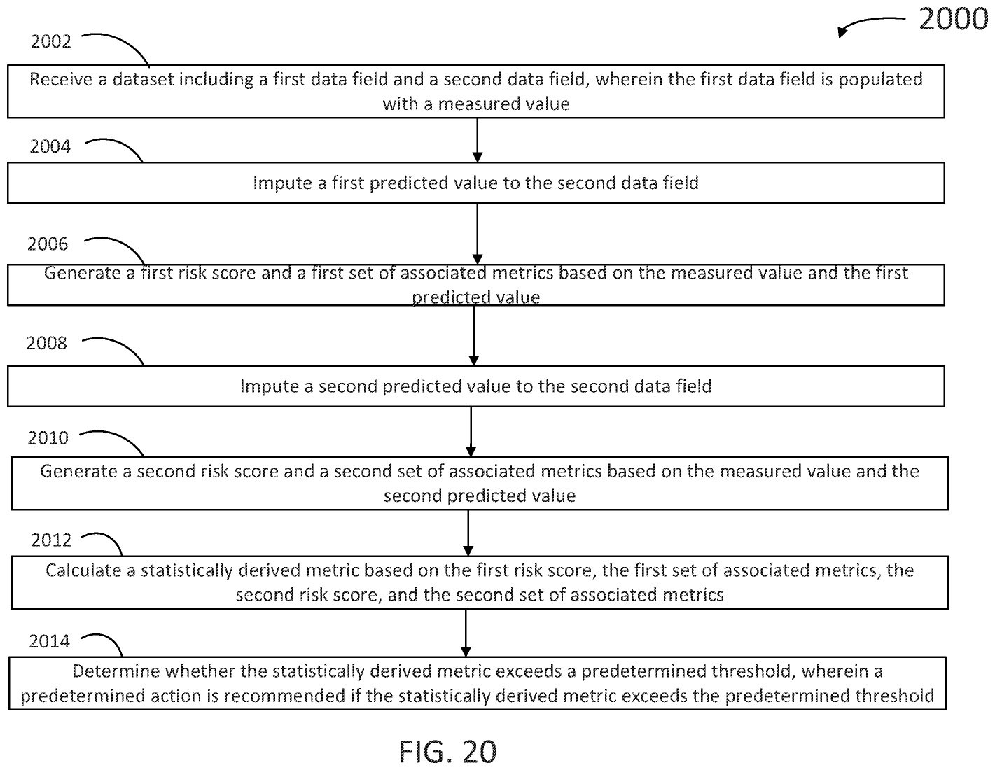

FIG. 20 is a flow chart illustrating steps in a method 2000 to perform a medical action on a patient based on multiple medical features received or imputed over a time sequence, in accordance with various embodiments. Method 2000 may be performed at least partially by any one of client devices coupled to one or more servers through a network (e.g., any one of servers 130 and any one of client devices 110, and network 150). For example, in accordance to various embodiments, the servers may host one or more medical devices or portable computer devices carried by medical or healthcare personnel. The client devices may be handled by a user such as a worker or other personnel in a healthcare facility, or a paramedic in an ambulance carrying a patient to the emergency room of a healthcare facility or hospital, an ambulance, or attending to a patient at a private residence or in a public location remote to the healthcare facility. At least some of the steps in method 2000 may be performed by a computer having a processor executing commands stored in a memory of the computer (e.g., processors 212 and memories 220). In accordance to various embodiments, the user may activate an application in the client device to access, through the network, a trigger logic engine in the server (e.g., application 222 and trigger logic engine 240). The trigger logic engine may include a modeling tool, a statistics tool, and an imputation tool to retrieve, supply, and process clinical data in real-time, and provide an action recommendation thereof (e.g., modeling tool 242, statistics tool 244, and imputation tool 246). Further, steps as disclosed in method 2000 may include retrieving, editing, and/or storing files in a database that is part of, or is communicably coupled to, the computer, using, inter-alia, a trigger logic engine (e.g., databases 252). Methods consistent with the present disclosure may include at least some, but not all, of the steps illustrated in method 2000, performed in a different sequence. Furthermore, methods consistent with the present disclosure may include at least two or more steps as in method 2000 performed overlapping in time, or almost simultaneously.

Step 2002 includes receiving a dataset including a first data field and a second data field, wherein the first data field is populated with a measured value.

Step 2004 includes imputing a first predicted value to the second data field.

Step 2006 includes generating a first risk score and a first set of associated metrics based on the measured value and the first predicted value.

Step 2008 includes imputing a second predicted value to the second data field.

Step 2010 includes generating a second risk score and a second set of associated metrics based on the measured value and the second predicted value.

Step 2012 includes calculating a statistically derived metric based on the first risk score, the first set of associated metrics, the second risk score, and the second set of associated metrics.

Step 2014 includes determining whether the statistically derived metric exceeds a predetermined threshold, wherein a predetermined action is recommended if the statistically derived metric exceeds the predetermined threshold.

### Hardware Overview

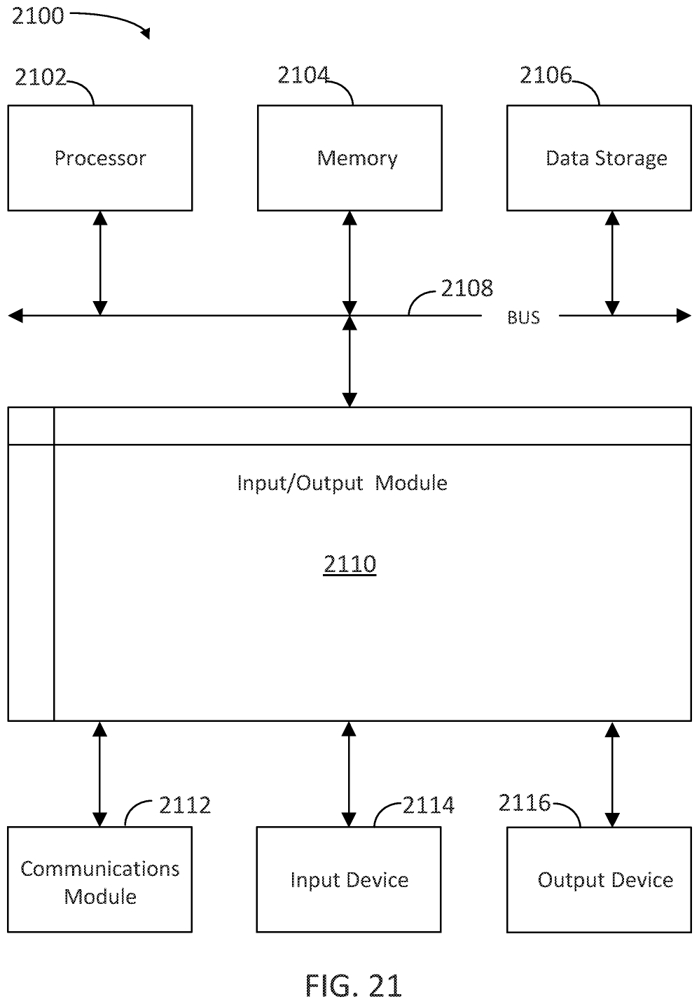

FIG. 21 is a block diagram illustrating an exemplary computer system 2100 with which the client device 110 and server 130 of FIGS. 1 and 2, and the methods of FIGS. 18-20 can be implemented. In certain aspects, the computer system 2100 may be implemented using hardware or a combination of software and hardware, either in a dedicated server, or integrated into another entity, or distributed across multiple entities.

Computer system 2100 (e.g., client device 110 and server 130) includes a bus 2108 or other communication mechanism for communicating information, and a processor 2102 (e.g., processors 212) coupled with bus 2108 for processing information. By way of example, the computer system 2100 may be implemented with one or more processors 2102. Processor 2102 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer system 2100 can include, in addition to hardware, code that creates an execution environment for the computer program in question, e.g., code that constitutes processor firmware, a protocol stack, a database management system, an operating system, or a combination of one or more of them stored in an included memory 2104 (e.g., memories 220), such as a Random Access Memory (RAM), a flash memory, a Read-Only Memory (ROM), a Programmable Read-Only Memory (PROM), an Erasable PROM (EPROM), registers, a hard disk, a removable disk, a CD-ROM, a DVD, or any other suitable storage device, coupled to bus 2108 for storing information and instructions to be executed by processor 2102. The processor 2102 and the memory 2104 can be supplemented by, or incorporated in, special purpose logic circuitry.

The instructions may be stored in the memory 2104 and implemented in one or more computer program products, i.e., one or more modules of computer program instructions encoded on a computer-readable medium for execution by, or to control the operation of, the computer system 2100, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. Memory 2104 may also be used for storing temporary variable or other intermediate information during execution of instructions to be executed by processor 2102.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

Computer system 2100 further includes a data storage device 2106 such as a magnetic disk or optical disk, coupled to bus 2108 for storing information and instructions. Computer system 2100 may be coupled via input/output module 2110 to various devices. Input/output module 2110 can be any input/output module. Exemplary input/output modules 2110 include data ports such as USB ports. The input/output module 2110 is configured to connect to a communications module 2112. Exemplary communications modules 2112 (e.g., communications modules 218) include networking interface cards, such as Ethernet cards and modems. In certain aspects, input/output module 2110 is configured to connect to a plurality of devices, such as an input device 2114 (e.g., input device 214) and/or an output device 2116 (e.g., output device 216). Exemplary input devices 2114 include a keyboard and a pointing device, e.g., a mouse or a trackball, by which a user can provide input to the computer system 2100. Other kinds of input devices 2114 can be used to provide for interaction with a user as well, such as a tactile input device, visual input device, audio input device, or brain-computer interface device. For example, feedback provided to the user can be any form of sensory feedback, e.g., visual feedback, auditory feedback, or tactile feedback; and input from the user can be received in any form, including acoustic, speech, tactile, or brain wave input. Exemplary output devices 2116 include display devices, such as an LCD (liquid crystal display) monitor, for displaying information to the user.

According to one aspect of the present disclosure, the client device 110 and server 130 can be implemented using a computer system 2100 in response to processor 2102 executing one or more sequences of one or more instructions contained in memory 2104. Such instructions may be read into memory 2104 from another machine-readable medium, such as data storage device 2106. Execution of the sequences of instructions contained in main memory 2104 causes processor 2102 to perform the process steps described herein. One or more processors in a multi-processing arrangement may also be employed to execute the sequences of instructions contained in memory 2104. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. The communication network (e.g., network 150) can include, for example, any one or more of a LAN, a WAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

Computer system 2100 can include clients and servers. A client and server are generally remote from each other and typically interact through a communication network. The relationship of client and server arises by virtue of computer programs running on the respective computers and having a client-server relationship to each other. Computer system 2100 can be, for example, and without limitation, a desktop computer, laptop computer, or tablet computer. Computer system 2100 can also be embedded in another device, for example, and without limitation, a mobile telephone, a PDA, a mobile audio player, a Global Positioning System (GPS) receiver, a video game console, and/or a television set top box.

The term “machine-readable storage medium” or “computer-readable medium” as used herein refers to any medium or media that participates in providing instructions to processor 2102 for execution. Such a medium may take many forms, including, but not limited to, non-volatile media, volatile media, and transmission media. Non-volatile media include, for example, optical or magnetic disks, such as data storage device 2106. Volatile media include dynamic memory, such as memory 2104. Transmission media include coaxial cables, copper wire, and fiber optics, including the wires that include bus 2108. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter effecting a machine-readable propagated signal, or a combination of one or more of them.

As used herein, the phrase “at least one of” preceding a series of items, with the terms “and” or “or” to separate any of the items, modifies the list as a whole, rather than each member of the list (i.e., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

To the extent that the term “include,” “have,” or the like is used in the description or the claims, such term is intended to be inclusive in a manner similar to the term “comprise” as “comprise” is interpreted when employed as a transitional word in a claim. The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products. Other variations are within the scope of the following claims.

### Recitation of Embodiments

1. A method for making dynamic risk predictions is provided, the method including: receiving a dataset including a first data field and a second data field, wherein the first data field is populated with a measured value; imputing a first predicted value to the second data field; generating a first risk score and a first set of associated metrics based on the measured value and the first predicted value; imputing a second predicted value to the second data field; generating a second risk score and a second set of associated metrics based on the measured value and the second predicted value; calculating a statistically derived metric based on the first risk score, the first set of associated metrics, the second risk score, and the second set of associated metrics; and determining whether the statistically derived metric exceeds a predetermined threshold, wherein a predetermined action is recommended if the statistically derived metric exceeds the predetermined threshold.

2. The method of embodiment 1, wherein generating the first set of associated metrics includes determining a variability induced in the first risk score by a sampling variability in a within standard deviation value.

3. The method of embodiments 1 or 2, wherein calculating the statistically derived metric includes calculating a standard deviation of the first risk score and the second risk score, referred to as the between standard deviation.

4. The method of any one of embodiments 1 through 3, wherein calculating the statistically derived metric includes calculating a total standard deviation that includes a between standard deviation and a within standard deviation value derived from the first risk score, second risk score, or mathematical combination of both.

5. The method of any one of embodiments 1 through 4, wherein calculating the statistically derived metric includes selecting a first risk score or second risk score or mathematical combination of both, total standard deviation, between standard deviation, or a within standard deviation value derived from the first risk score, second risk score, or mathematical combination of both.

6. The method of any one of embodiments 1 through 5, wherein calculating the statistically derived metric includes determining a ratio between any two of the following: a first risk score or second risk score or mathematical combination of both, a total standard deviation, between standard deviation, or a within standard deviation value derived from the first risk score, second risk score, or mathematical combination of both.

7. The method of any one of embodiments 1 through 6, wherein calculating the predetermined threshold includes evaluating a polynomial function of the first risk score or the second risk score and comparing an output of that function to a total standard deviation, between standard deviation, or a within standard deviation value derived from the first risk score, second risk score, or mathematical combination of both.

8. The method of any one of embodiments 1 through 7, wherein the first set of associated metrics corresponds to a first collection time, the second set of associated metrics corresponds to a second collection time, and determining whether the statistically derived metric exceeds the predetermined threshold includes using a stateful logic after the first collection time and the second collection time.

9. The method of any one of embodiments 1 through 8, wherein the first set of associated metrics corresponds to a first collection time, the second set of associated metrics corresponds to a second collection time, and determining whether the statistically derived metric exceeds the predetermined threshold includes using a stateless logic after one of the first collection time or the second collection time.

10. The method of any one of embodiments 1 through 9, wherein imputing a first predicted value to the second data field includes determining the first predicted value based on the measured value and a conditional rule relating the first data field to the second data field.

11. A system is provided, the system including a memory configured to store instructions and one or more processors communicatively coupled to the memory and configured to execute instructions and cause the system to: receive a dataset including a first data field and a second data field, wherein the first data field is populated with a measured value; impute a first predicted value to the second data field; generate a first risk score and a first set of associated metrics based on the measured value and the first predicted value; impute a second predicted value to the second data field; generate a second risk score and a second set of associated metrics based on the measured value and the second predicted value; calculate a statistically derived metric based on the first risk score, the first set of associated metrics, the second risk score, and the second set of associated metrics; and determine whether the statistically derived metric exceeds a predetermined threshold, wherein a predetermined action is recommended if the statistically derived metric exceeds the predetermined threshold, wherein generating the first set of associated metrics includes determining a variability induced in the first risk score by the first predicted value in a between standard deviation value.

12. The system of embodiment 11, wherein to generate the first set of associated metrics the one or more processors execute instructions to determine a variability induced in the first risk score by a sampling variability in a within standard deviation.

13 The system of embodiments 11 or 12, wherein to generate the first set of associated metrics the one or more processors execute instructions to determine a total standard deviation that includes a between standard deviation and a within standard deviation.

14. The system of any one of embodiments 11 through 13, wherein to calculate the statistically derived metric the one or more processors execute instructions to select a first risk score or second risk score or mathematical combination of both, total standard deviation, between standard deviation, or a within standard deviation value derived from the first risk score, second risk score, or mathematical combination of both.

15. The system of any one of embodiments 11 through 14, wherein to calculate the statistically derived metric the one or more processors execute instructions to determine a ratio between any two of the following: a first risk score or second risk score or mathematical combination of both, a total standard deviation, between standard deviation, or a within standard deviation value derived from the first risk score, second risk score, or mathematical combination of both.

16. A non-transitory, computer readable medium storing instructions which, when executed by a computer, cause the computer to perform a method is provided, the method including: receiving a dataset including a first data field and a second data field, wherein the first data field is populated with a measured value; imputing a first predicted value to the second data field; generating a first risk score and a first set of associated metrics based on the measured value and the first predicted value; imputing a second predicted value to the second data field; generating a second risk score and a second set of associated metrics based on the measured value and the second predicted value; calculating a statistically derived metric based on the first risk score, the first set of associated metrics, the second risk score, and the second set of associated metrics; and determining whether the statistically derived metric exceeds a predetermined threshold, wherein a predetermined action is recommended if the statistically derived metric exceeds the predetermined threshold, wherein generating the first set of associated metrics includes determining a variability induced in the first risk score by the first predicted value in a between standard deviation value and in a within standard deviation value.

17. The non-transitory, computer readable medium of embodiment 16 wherein, in the method, calculating the statistically derived metric includes evaluating a polynomial function of the first risk score or the second risk score and comparing an output of that function to a total standard deviation, between standard deviation, or a within standard deviation value derived from the first risk score, second risk score, or mathematical combination of both.

18. The non-transitory, computer readable medium of embodiments 16 or 17, wherein the first set of associated metrics corresponds to a first collection time, the second set of associated metrics corresponds to a second collection time, and determining whether the statistically derived metric exceeds the predetermined threshold includes using a stateful logic after the first collection time and the second collection time.

19. The non-transitory, computer readable medium of any one of embodiments 16 through 18, wherein the first set of associated metrics corresponds to a first collection time, the second set of associated metrics corresponds to a second collection time, and determining whether the statistically derived metric exceeds the predetermined threshold includes using a stateless logic after one of the first collection time or the second collection time.

20. The non-transitory, computer readable medium of any one of embodiments 16 through 19, wherein imputing a first predicted value to the second data field includes determining the first predicted value based on the measured value and a conditional rule relating the first data field to the second data field.

What is claimed is:

1. A method for making dynamic risk predictions, comprising:
receiving a dataset comprising a first data field and a second data field, wherein the first data field is populated with a measured value;
imputing a first predicted value to the second data field;
generating a first risk score and a first set of associated metrics based on the measured value and the first predicted value;
imputing a second predicted value to the second data field;
generating a second risk score and a second set of associated metrics based on the measured value and the second predicted value;
calculating a statistically derived metric based on the first risk score, the first set of associated metrics, the second risk score, and the second set of associated metrics; and
determining whether the statistically derived metric exceeds a predetermined threshold, wherein a predetermined action is recommended if the statistically derived metric exceeds the predetermined threshold.

2. The method of claim 1, wherein generating the first set of associated metrics comprises determining a variability induced in the first risk score by a sampling variability in a within standard deviation value.

3. The method of claim 1, wherein calculating the statistically derived metric comprises calculating a standard deviation of the first risk score and the second risk score, referred to as the between standard deviation.

4. The method of claim 1, wherein calculating the statistically derived metric comprises calculating a total standard deviation that includes a between standard deviation and a within standard deviation value derived from the first risk score, second risk score, or mathematical combination of both.

5. The method of claim 1, wherein calculating the statistically derived metric comprises selecting a first risk score or second risk score or mathematical combination of both, total standard deviation, between standard deviation, or a within standard deviation value derived from the first risk score, second risk score, or mathematical combination of both.

6. The method of claim 1, wherein calculating the statistically derived metric comprises determining a ratio between any two of the following: a first risk score or second risk score or mathematical combination of both, a total standard deviation, between standard deviation, or a within standard deviation value derived from the first risk score, second risk score, or mathematical combination of both.

7. The method of claim 1, wherein calculating the predetermined threshold comprises evaluating a polynomial function of the first risk score or the second risk score and comparing an output of that function to a total standard deviation, between standard deviation, or a within standard deviation value derived from the first risk score, second risk score, or mathematical combination of both.

8. The method of claim 1, wherein the first set of associated metrics corresponds to a first collection time, the second set of associated metrics corresponds to a second collection time, and determining whether the statistically derived metric exceeds the predetermined threshold comprises using a stateful logic after the first collection time and the second collection time.

9. The method of claim 1, wherein the first set of associated metrics corresponds to a first collection time, the second set of associated metrics corresponds to a second collection time, and determining whether the statistically derived metric exceeds the predetermined threshold comprises using a stateless logic after one of the first collection time or the second collection time.

10. The method of claim 1, wherein imputing a first predicted value to the second data field comprises determining the first predicted value based on the measured value and a conditional rule relating the first data field to the second data field.

11. A system, comprising:
a memory configured to store instructions; and
one or more processors communicatively coupled to the memory and configured to execute instructions and cause the system to:
receive a dataset comprising a first data field and a second data field, wherein the first data field is populated with a measured value;
impute a first predicted value to the second data field;
generate a first risk score and a first set of associated metrics based on the measured value and the first predicted value;
impute a second predicted value to the second data field;
generate a second risk score and a second set of associated metrics based on the measured value and the second predicted value;
calculate a statistically derived metric based on the first risk score, the first set of associated metrics, the second risk score, and the second set of associated metrics; and
determine whether the statistically derived metric exceeds a predetermined threshold, wherein a predetermined action is recommended if the statistically derived metric exceeds the predetermined threshold, wherein generating the first set of associated metrics comprises determining a variability induced in the first risk score by the first predicted value in a between standard deviation value.

12. The system of claim 11, wherein to generate the first set of associated metrics the one or more processors execute instructions to determine a variability induced in the first risk score by a sampling variability in a within standard deviation.

13. The system of claim 11, wherein to generate the first set of associated metrics the one or more processors execute instructions to determine a total standard deviation that includes a between standard deviation and a within standard deviation.

14. The system of claim 11, wherein to calculate the statistically derived metric the one or more processors execute instructions to select a first risk score or second risk score or mathematical combination of both, total standard deviation, between standard deviation, or a within standard deviation value derived from the first risk score, second risk score, or mathematical combination of both.

15. The system of claim 11, wherein to calculate the statistically derived metric the one or more processors execute instructions to determine a ratio between any two of the following: a first risk score or second risk score or mathematical combination of both, a total standard deviation, between standard deviation, or a within standard deviation value derived from the first risk score, second risk score, or mathematical combination of both.

16. A non-transitory, computer readable medium storing instructions which, when executed by a computer, cause the computer to perform a method, the method comprising:
receiving a dataset comprising a first data field and a second data field, wherein the first data field is populated with a measured value;
imputing a first predicted value to the second data field;
generating a first risk score and a first set of associated metrics based on the measured value and the first predicted value;
imputing a second predicted value to the second data field;
generating a second risk score and a second set of associated metrics based on the measured value and the second predicted value;
calculating a statistically derived metric based on the first risk score, the first set of associated metrics, the second risk score, and the second set of associated metrics; and
determining whether the statistically derived metric exceeds a predetermined threshold, wherein a predetermined action is recommended if the statistically derived metric exceeds the predetermined threshold, wherein generating the first set of associated metrics comprises determining a variability induced in the first risk score by the first predicted value in a between standard deviation value and in a within standard deviation value.

17. The non-transitory, computer readable medium of claim 16 wherein, in the method, calculating the statistically derived metric comprises evaluating a polynomial function of the first risk score or the second risk score and comparing an output of that function to a total standard deviation, between standard deviation, or a within standard deviation value derived from the first risk score, second risk score, or mathematical combination of both.

18. The non-transitory, computer readable medium of claim 16, wherein the first set of associated metrics corresponds to a first collection time, the second set of associated metrics corresponds to a second collection time, and determining whether the statistically derived metric exceeds the predetermined threshold comprises using a stateful logic after the first collection time and the second collection time.

19. The non-transitory, computer readable medium of claim 16, wherein the first set of associated metrics corresponds to a first collection time, the second set of associated metrics corresponds to a second collection time, and determining whether the statistically derived metric exceeds the predetermined threshold comprises using a stateless logic after one of the first collection time or the second collection time.

20. The non-transitory, computer readable medium of claim 16, wherein imputing a first predicted value to the second data field comprises determining the first predicted value based on the measured value and a conditional rule relating the first data field to the second data field.
